(19) United States
(12) Patent Application Publication
(10) Pub. No.: US 2013/0065238 A1
(43) Pub. Date: Mar. 14, 2013

(54) SYNGAP1 DYSFUNCTIONS AND USES THEREOF IN DIAGNOSTIC AND THERAPEUTIC APPLICATIONS FOR MENTAL RETARDATION

(71) Applicants: Centre Hospitalier Universitaire Sainte-Justine, Montreal (CA); Centre Hospitalier De L'Universite De Montreal, Montreal (CA)

(72) Inventors: Jacques MICHAUD, Montreal (CA); Fadi HAMDAN, Baie D'Urfe (CA); Guy ROULEAU, Montreal (CA); Julie GAUTHIER, Montreal (CA)

(73) Assignees: CENTRE HOSPITALIER DE L'UNIVERSITE DE MONTREAL, Montreal, QC (CA); CENTRE HOSPITALIER UNIVERSITAIRE SAINTE-JUSTINE, Montreal, QC (CA)

(21) Appl. No.: 13/673,034

(22) Filed: Nov. 9, 2012

Related U.S. Application Data
(63) Continuation of application No. 13/127,733, filed on May 19, 2011.
(60) Provisional application No. 61/112,345, filed on Nov. 7, 2008.

(51) Int. Cl. C12Q 1/68; G01N 33/53; C07K 17/00; C07H 21/00; C07K 14/47; C07K 16/18; G01N 33/566; C12Q 1/02

(52) U.S. Cl. 435/6.11; 435/6.12; 435/7.1; 435/29; 536/24.31; 530/350; 530/387.9; 530/391.1

(57) ABSTRACT

The invention identifies Syngap1 dysfunctions as causative of mental retardation. Described are methods of detecting mental retardation and methods of detecting non-syndromic mental retardation (NSMR) in a human subject. Particular methods comprise sequencing a human subject's genomic DNA for comparison with a control sequence from an unaffected individual. Also described are probes, kits, antibodies and isolated mutated Syngap1 proteins.

### FIELD OF THE INVENTION

The invention relates to the field of genetic diseases. More particularly, it relates to the identification of Syngap1 dysfunctions as causative of mental retardation.

### BACKGROUND OF THE INVENTION

Mental retardation (MR) is the most frequent severe handicap of children, affecting 1-3% of the population. Most MR patients have the non-syndromic form, which is characterized by the absence of associated morphological, radiological or metabolic features. However, sometimes the separation between both forms of the disease could be very subtle (Chelly et al., 2006 Eur J Hum Genet 14(6), 701-13).

The genetics of non-syndromic MR (NSMR) remains poorly understood. Linkage and cytogenetic analyses have led to the identification of 29 X-linked and 5 autosomal recessive NSMR genes, which, together, explain less than 10% of cases (Ropers et al., 2005 Nat Rev Genet 6 (1): 56-57; Basel-Vanagaite et al. 2007 Clin Genet 72(3): 167-74). Moreover, autosomal dominant NSMR genes have not yet been identified. There is thus a need for the identification of the genes and causes (e.g. monoallelic dysfunctions, de novo genetic dysfunctions, point mutations, etc.) associated with NSMR.

SYNGAP stands for Synaptic GTPase Activating Protein. Syngap1 is a GTPase activating protein (GAP) that is selectively expressed in the brain and that is a component of the NMDAR complex (Chen et al., 1998 Neuron 20 (5): 895-904). The human gene is found on chromosome 6 and there are at least three different isoforms of the proteins which are known in humans (see NCBI accession numbers NM_{—}006772.2, NM_{—}001130066 and AL713634). The rat sequence is described in U.S. Pat. No. 6,723,838. Although Syngap1 appears to have an essential role during early postnatal development, its function (or dysfunctions thereof) had not been associated, with mental retardation problems. Such an association was made by the present inventors and published recently (Hamdan et al., N Engl J Med. 2009, 360(6):599-605).

The present inventors have now demonstrated that the Syngap1 gene is a causal gene for a large fraction of non-syndromic mental retardation (NSMR), thereby leading to the development of a variety of methods for the screening of the disease, for diagnosis of the disease and for developing therapies for treatment of disease. Since the separation between syndromic and non-syndromic forms of mental retardation could be sometimes very subtle and in some cases mutations in the same gene could lead to either form of the disease (depending on the severity of the mutation and the genetic background of the affected individual), the methods covered for SYNGAP1 in this patent applies to mental retardation in general.

### BRIEF SUMMARY OF THE INVENTION

The invention relates to the identification of Syngap1 dysfunctions as causative of mental retardation.

The invention concerns methods of detecting mental retardation and methods of detecting non-syndromic mental retardation (NSMR) in a human subject. In some embodiments, the methods comprise assessing a biological sample from the subject for identifying Syngap1 dysfunctions. Preferably, the biological sample comprises nucleic acid molecules and the assessment comprises analysing the nucleic acid molecules for the presence or absence of a pathogenic mutation in a Syngap1 encoding nucleic acid molecule.

One particular aspect of the invention relates to a method of diagnosing mental retardation (MR) in a human subject. The method comprises assaying a biological sample from the human subject for detecting the presence or absence of a pathogenic Syngap1 dysfunction. In one embodiment, the pathogenic Syngap1 dysfunction comprises a pathogenic mutation in a Syngap1 gene comprising SEQ ID NO: 7. In another embodiment the presence of the pathogenic Syngap1 dysfunction is characterized by a de novo genomic mutation in Syngap1. In one embodiment the dysfunction is a truncating mutation causing expression of a truncated Syngap1 protein comprising an amino acid sequence other than SEQ ID NO: 2, SEQ ID NO: 4, or SEQ ID NO: 6. In another embodiment the truncated Syngap1 protein comprises an amino acid sequence selected from the group consisting of SEQ ID NO:8, SEQ ID NO:9 or SEQ ID NO:10. In a preferred embodiment the biological sample comprises sequencing nucleic acids obtained from the subject, and those nucleic acids comprise at least a portion of a Syngap1 gene as set forth in SEQ ID NO:7.

Another aspect of the invention concerns a method of diagnosing mental retardation (MR) in a human subject. The method comprising: (a) obtaining from a human subject a biological sample comprising genomic DNA; (b) sequencing the genomic DNA for obtaining a sequence of one or more regions responsible in expression of Syngap1; (c) comparing the sequence obtained at (b) with a corresponding control sequence from an unaffected individual. The comparison at (c) allows identification of the presence or absence of a pathogenic Syngap1 genomic mutation.

The methods of the inventions are useful for detecting mental retardation in general, and more particularly non-syndromic mental retardation (NSMR). Therefore a more particular aspect concerns a method for diagnosing non-syndromic mental retardation (NSMR) in a human subject. The method comprises detecting in a nucleic acid sample obtained from the subject the presence or absence of a de novo genomic mutation in a Syngap1 gene comprising SEQ ID NO:7. In one embodiment the de novo genomic mutation is a heterologous mutation. In preferred embodiments the de novo genomic mutation is a nonsense mutation or a frameshift mutation. Examples of detection include sequencing DNA or RNA molecules from the subject.

In one particular embodiment, the method for diagnosing non-syndromic mental retardation (NSMR) in a human subject comprises: (a) obtaining from the subject a biological sample having DNA; (b) sequencing regions of the subject's DNA encoding a Syngap1 protein; and (c) comparing the sequence obtained at (b) with a corresponding sequence from an unaffected individual (e.g. a parent) for identifying a pathogenic Syngap1 mutation; wherein the identification of a pathogenic Syngap1 mutation is correlated with NSMR.

One particular aspect of the invention relates to an isolated nucleic acid molecule comprising a sequence encoding a mutated Syngap1 protein. Another aspect relates to nucleic acid probes such as probes hybridizing specifically to a nucleic acid molecule comprising a genomic mutation in a Syngap1 gene of SEQ ID NO: 7, or hybridizing specifically to a complementary strand thereof.

Another aspect relates to an isolated mutated Syngap1 protein. Another related aspect concerns a fragment of the nucleic acid molecule or of the mutated Syngap1 protein, the fragment comprising a dysfunction (e.g. a pathogenic Syngap1 dysfunction). The invention also relates to monoclonal or polyclonal antibodies which specifically recognize Syngap1 mutated proteins.

A related aspect concerns a solid support comprising a compound (e.g. a nucleic acid probe or an antibody as defined herein) for identifying a pathogenic Syngap1 dysfunction in a human subject, wherein the dysfunction is responsible for mental retardation.

The invention also concerns kits for detecting the presence or absence of a mutant Syngap1 nucleic acid molecule in a biological sample. In one embodiment, the kit comprises a user manual or instructions and at least one of: (i) a nucleic acid probe hybridizing specifically to a nucleic acid molecule comprising a genomic mutation in a Syngap1 gene comprising SEQ ID NO: 7; (ii) a nucleic acid probe hybridizing specifically to a complementary strand of the nucleic acid molecule according to (i); (iii) a monoclonal or polyclonal antibody as defined herein; and (iv) a compound for measuring the amount and/or activity of a Syngap1 protein in the biological sample.

The invention further relates to a screening method for identifying suitable drugs for restoring Syngap1 function. In one embodiment, the screening method comprises contacting a cell or animal having a mutant Syngap1 gene with a compound to be tested; and assessing activity of the compound on Syngap1 activity and/or levels.

Methods for treating, improving, or alleviating mental retardation in a human subject are also the subject of the present invention. According to one embodiment, the method comprises administering to the subject a therapeutically effective amount of a normal Syngap1 protein or a therapeutically effective amount of a compound compensating for a pathogenic Syngap1 mutation in a human subject. According to another embodiment, the method comprises administering to a human subject having a defective Syngap1 protein activity a therapeutically effective amount of a compound that restores Syngap1 activity to a desirable level. According to a further embodiment, the method comprises administering to the subject a therapeutically effective amount of a compound inhibiting or activating signaling pathways regulated by Syngap1. Preferably, the therapeutic compounds according to the invention are capable of crossing the blood brain barrier (BBB). Compounds that may be therapeutically effective include, but are not limited to, compounds that modify the activity of ribosomes, inhibitors or effectors of RAS, and inhibitors or effectors of RAP.

A related aspect of the invention is a method of gene therapy for mental retardation in a human subject, comprising the delivery of a nucleic acid molecule which includes a sequence corresponding to a normal Syngap1 DNA sequence encoding a functional Syngap1 protein.

Additional aspects, advantages and features of the present invention will become more fully understood from the detailed description given herein and from the accompanying drawings, which are exemplary and should not be interpreted as limiting the scope of the invention.

### BRIEF DESCRIPTION OF THE FIGURES

FIG. 1 shows the mRNA sequence (SEQ ID NO:1) and the corresponding protein sequence (SEQ ID NO:2) of SYNGAP1 isoform 1. The sequences are based on NCBI reference sequence #NM_{—}006772.2 (mRNA) and NP_{—}006763.2 (protein). Small caps indicate untranslated regions. The accession number in the Uniprot database for the protein sequence is Q96PV0 (under isoform1).

FIG. 2 shows the mRNA sequence (SEQ ID NO:3) and the corresponding protein sequence (SEQ ID NO:4) of SYNGAP1 isoform 2. The sequences are based on NCBI reference sequence #NM_{—}001130066 (mRNA) and #NP_{—}001123538.1 (protein). Small caps indicate untranslated regions.

FIG. 3 shows the mRNA sequence (SEQ ID NO:5) and the corresponding protein sequence (SEQ ID NO:6) of SYNGAP1 isoform 3. The sequences are based on the first 1149 bp of the coding sequence reported in NCBI Refseq #NM_{—}006772.2 plus all the nucleotide sequence reported in NCBI Genbank accession #AL713634. Small caps indicate untranslated regions. The accession number in the Uniprot database for the protein sequence is Q96PV0 (under isoform2).

FIG. 4 shows SEQ ID NO: 7 which corresponds to genomic sequence of SYNGAP1 genomic sequence from hg18 assembly. The reference sequence is NCBI NM_{—}006772. Shown are exons (large caps) and introns (small caps) for isoform 1. Position: chr6:33495825-33529444. Band: 6p21.32. Genomic Size: 33620. Strand: +.

FIG. 5 shows the amino acid sequence of polypeptides resulting from de novo mutations identified in three patients with non-syndromic mental retardation. SEQ ID NO: 8 is a mutated protein from patient 1 (K138X). SEQ ID NO: 9 is a mutated protein from patient 2 (R579X). SEQ ID NO: 10 is a mutated protein from patient 3 (L813RfsX22).

FIG. 6 is a schema summarizing the results obtained in the course of identifying de novo SYNGAP1 mutations in three different NSMR patients. (A) Localization of de novo SYNGAP1 mutations identified in NSMR patients. Amino acid positions are based on the Refseq #NP_{—}006763 (from NM_{—}006772) (isoform 1: 1343 amino acids). The various predicted functional domains are highlighted: PH, pleckstrin homology domain (pos. 150-251), C2 domain (pos. 263-362), RASGAP (pos. 392-729), SH3 (pos. 785-815), CC domain (pos.1189-1262), T/SXV Type 1 PDZ-binding motif (“QTRV”; isoform 2), and CamKII binding (“GAAPGPPRHG”; isoform 3). The variable carboxyl-termini of the 3 SYNGAP1 isoforms shown here correspond to GenBank cDNA accession numbers: AB067525 for isoform 1; AK307888 for isoform 2; AL713634 for isoform 3. (B) Families with de novo mutations in SYNGAP1. Chromatograms corresponding to the SYNGAP1 sequence for each patient and her parents are shown. Wild type (WT) and mutant (MT) SYNGAP1 DNA sequences are shown along with the corresponding amino acids.

### DETAILED DESCRIPTION OF PREFERRED EMBODIMENTS

The invention identifies Syngap1 as a disease gene responsible for mental retardation. In aspects, Syngap1 is a causal gene for a large fraction of non-syndromic mental retardation (NSMR). Disruption of Syngap1 represents the first example of an autosomal dominant NSMR gene. Mutations in Syngap1 lead to the development of NSMR with or without epilepsy.

With the knowledge that mutations in the Syngap1 sequence are causal of NSMR, the genomic, cDNA and protein sequences thereof can be used in a variety of methods for the screening of the disease, for diagnosis of the disease, for developing therapies for treatment of disease, for developing pharmacological therapies of the disease and for the development of animal models of the disease. The knowledge of mutations causative of NSMR in the Syngap1 nucleic acid sequence is particularly beneficial DNA diagnosis and family counseling. It may also be useful for carrier detection where the mutation is recessive. Identification of Syngap1 as being causative of mental retardation in young children will help counselors in advising parents, and help in managing appropriate care for the affected children.

Prenatal diagnosis is useful to assess whether a fetus will be born with MR due to the presence of SYNGAP1 mutations. Prenatal diagnosis is also useful to determine whether a child will be born with a symptom or develop a symptom after birth selected from the group consisting of mental retardation with or without epilepsy. The invention encompasses the screening and diagnosis of any human or fetus that may have or be predisposed to have a Syngap1 gene mutation including but not limited to suspected MR subjects

### I. Definitions

As used herein and in the appended claims, the singular forms “a”, “an”, and “the” include plural referents unless the context clearly indicates otherwise. Thus, for example, reference to “a mutation” includes one or more of such mutations and reference to “the method” includes reference to equivalent steps and methods known to those of ordinary skill in the art that could be modified or substituted for the methods described herein.

“Syngap” or a “Syngap1” or “SYNGAP1” as used herein refers to a gene and the corresponding neuron-specific GTPase activating protein (GAP) that inhibits the activity of the small GTPases RAS and RAP. The Syngap1 protein is encoded by the Syngap1 gene that is found on chromosome 6 in humans. A more detailed overview of Syngap1 function and role is given hereinafter.

“Nucleic acid” or a “nucleic acid molecule” as used herein refers to any DNA or RNA molecule, either single or double stranded and, if single stranded, the molecule of its complementary sequence in either linear or circular form. The term encompasses modified and/or artificial nucleic acid molecules, including but not limited to, peptide nucleic acid (PNA) and locked nucleic acid (LNA). In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e. in cells or tissues). An “isolated nucleic acid” (either DNA or RNA) may further represent a molecule produced directly by biological or synthetic means and separated from other components present during its production.

A “vector” is a replicon, such as a plasmid, cosmid, bacmid, phage or virus, to which another genetic sequence or element (either DNA or RNA) may be attached so as to bring about the replication of the attached sequence or element.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). The present invention encompasses substantially pure Syngap1 materials (e.g., nucleic acids, oligonucleotides, proteins, fragments, mutants, etc.)

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

With respect to single-stranded nucleic acids, particularly oligonucleotides, the term “specifically hybridizing” or “hybridizing specifically” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single-stranded nucleic acid molecules of varying complementarity are well known in the art. For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5 with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. With regard to the nucleic acids of the current invention, a moderate stringency hybridization is defined as hybridization in 6×SSC, 5×Denhardt's solution, 0.5% SDS and 100 μg/ml denatured salmon sperm DNA at 42° C. and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

As used herein, the term “solid support” refers to any solid or stationary material to which reagents such as antibodies, antigens, and other test components can be attached. Examples of solid supports include, without limitation, microtiter plates (or dish), microscope (e.g. glass) slides, coverslips, beads, cell culture flasks, chips (for example, silica-based, glass, or gold chip), membranes, particles (typically solid; for example, agarose, sepharose, polystyrene or magnetic beads), columns (or column materials), and test tubes. Typically, the solid supports are water insoluble.

As used herein, an “instructional material” or a “user manual” includes a publication, a recording, a diagram, or any other medium of expression which can be used to communicate the usefulness of the compounds or compositions of the invention for performing a method according to the invention.

The term “mental retardation” as used herein, is broadly defined as a significantly sub-average general intellectual functioning that is accompanied by significant limitations in adaptive functioning. Mental retardation can be categorized as mild mental retardation (IQ from about 50-70) or as severe mental retardation (IQ less than 50).

As used herein, the term “biological sample” refers to a subset of the tissues of a biological organism, its cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen).

As used herein, the term “pathogenic Syngap1 dysfunction” is any alteration in Syngap1 biological activity which is causative of mental retardation in a human subject. This term encompasses any dysfunction or defect wherein state, quality, and/or levels of Syngap1 biological activity are impacted. In particular embodiment it more specifically refers to a pathogenic Syngap1 mutation, i.e. a mutation which alters function or expression of Syngap1 gene products.

A “mutation” is any alteration in a gene which alters function or expression of the gene products, such as mRNA and the encoded for protein. This include but is not limited to altering mutation, point mutation, truncation mutation, deletion mutation, frame-shift mutation, and null mutation, nonsense mutation, missense mutation, and a mutation affecting exon splicing (consensus splice sites).

Because the majority of disease causing pathogenic mutations are in the coding region and splice junctions of genes, preferred embodiments of the invention focuses on these regions. Nevertheless, the invention does not preclude the possibility of detecting the presence or absence of a pathogenic Syngap1 dysfunction by examining other regions including, but not limited to, regulatory elements (e.g. promoter, untranslated regions, other intronic splice sites) that could also disrupt SYNGAP1 production and function.

### II. Nucleic Acid Molecules

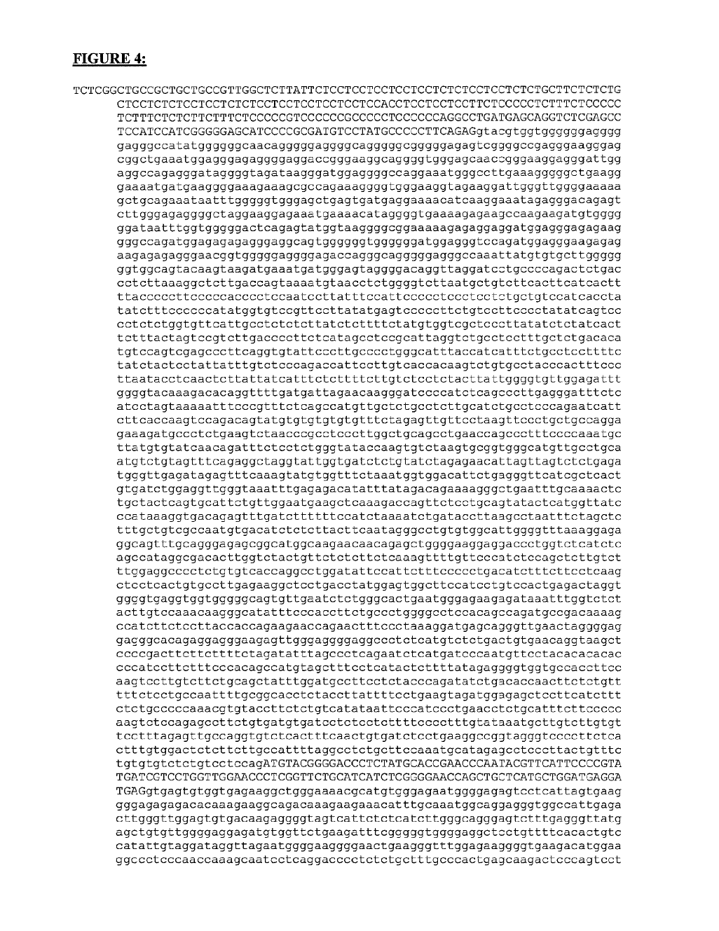

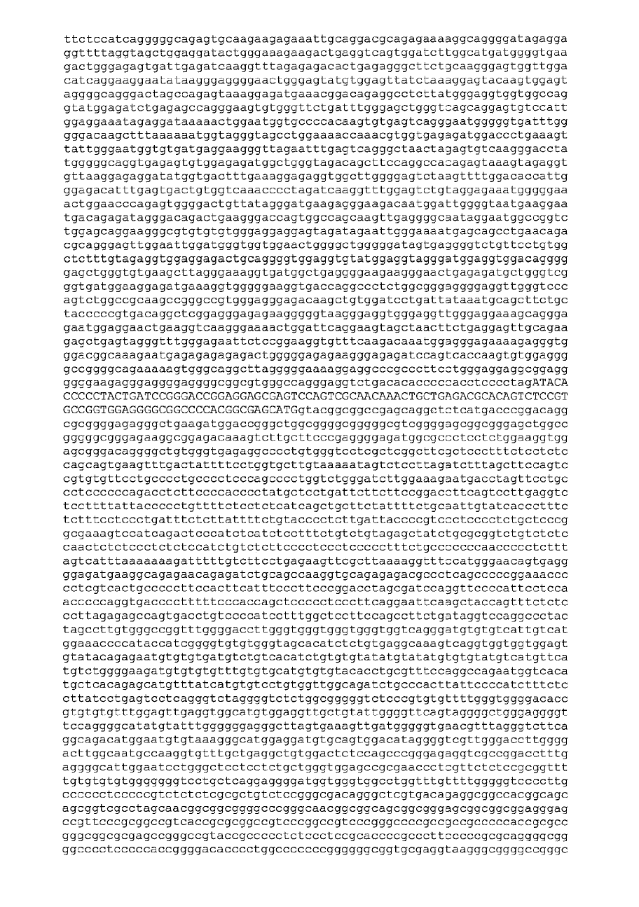

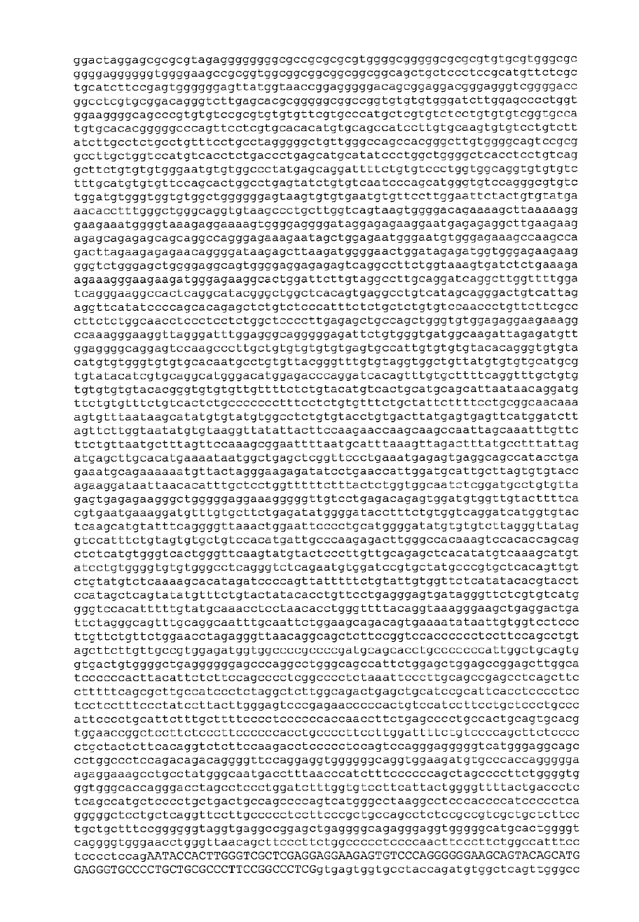

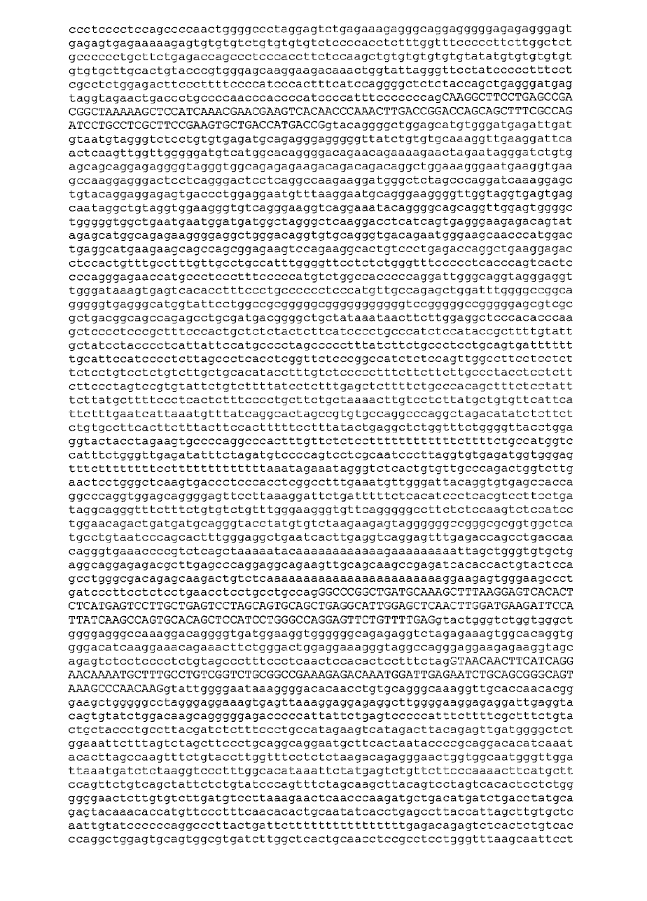

Syngap1 is a gene which is found in humans on chromosome 6, band 6p21.32. The genomic sequence of human Syngap1 is shown in FIG. 4 (represented as SEQ ID NO:7).

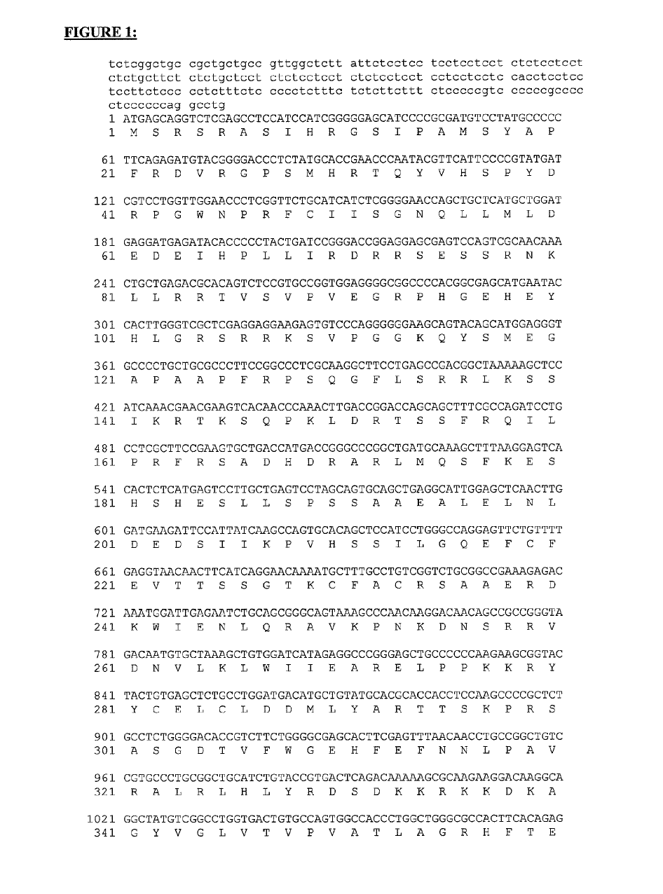

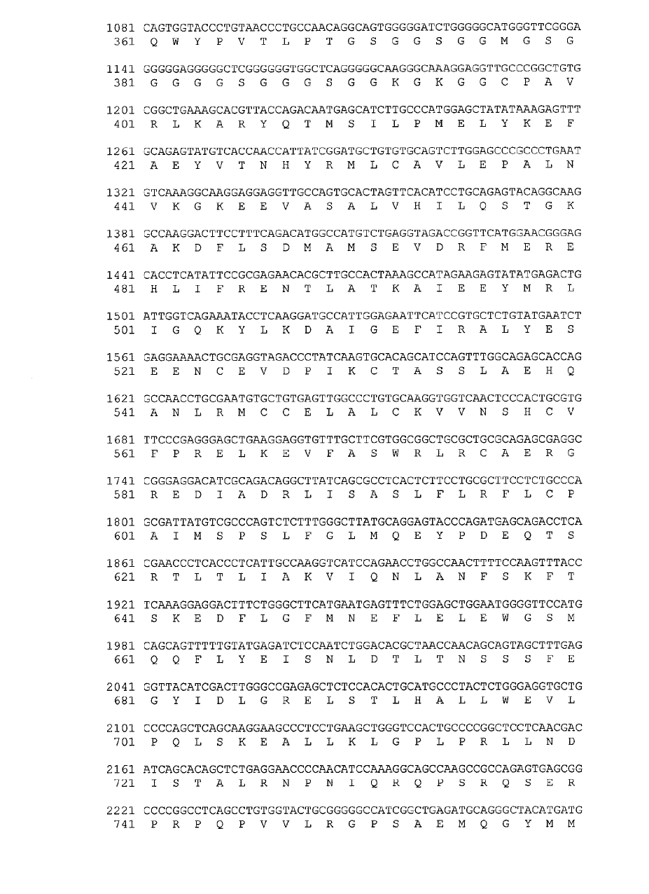

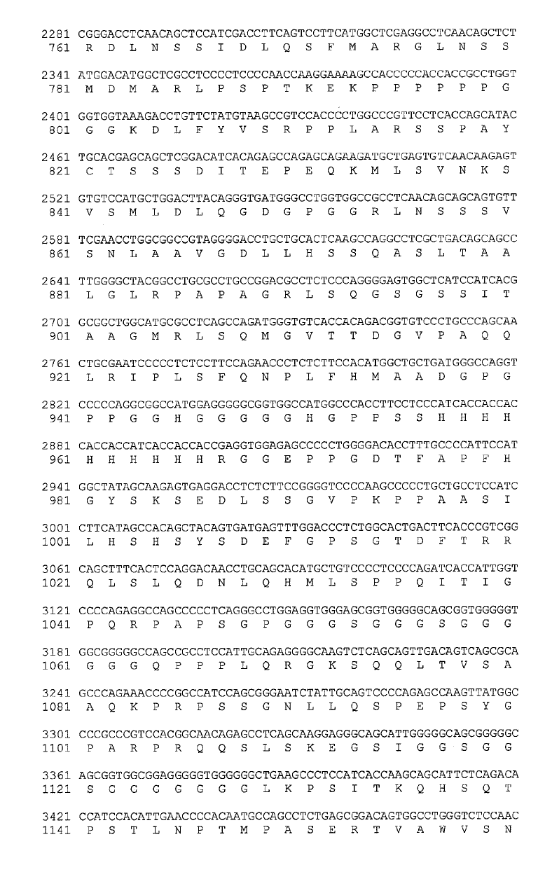

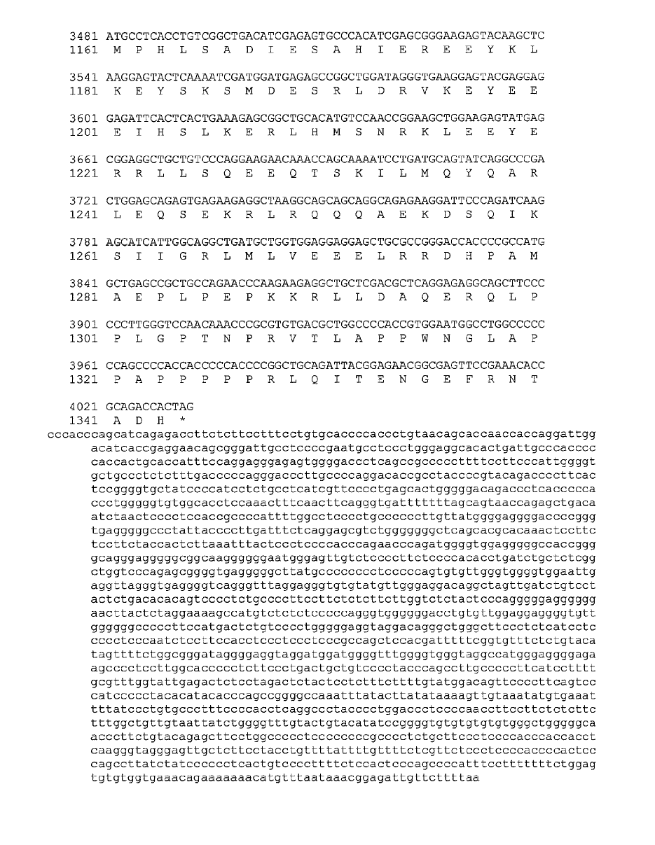

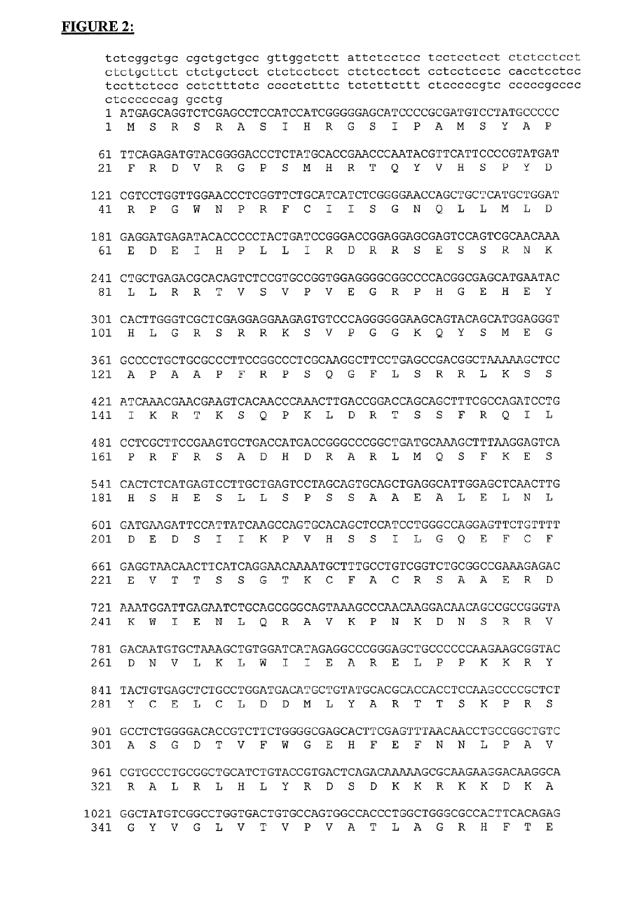

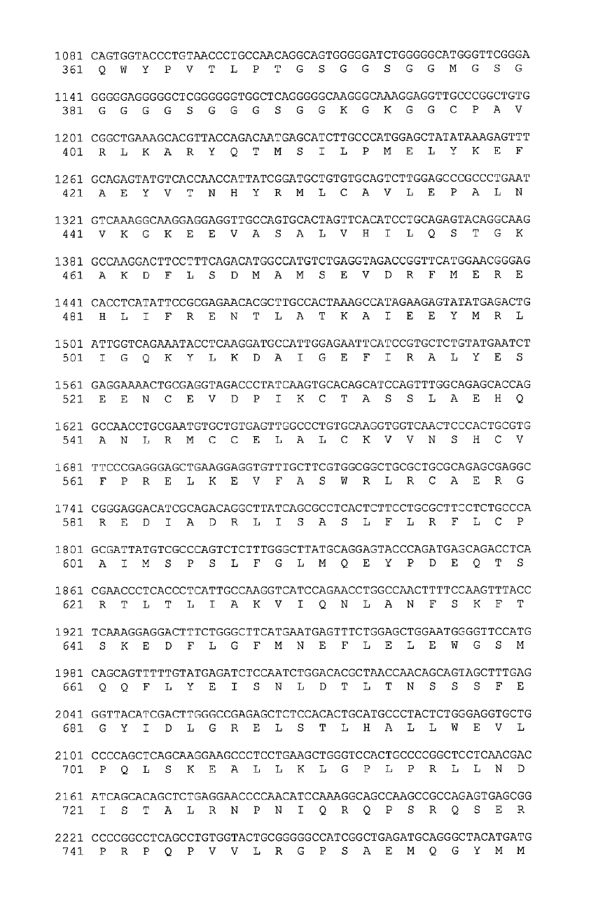

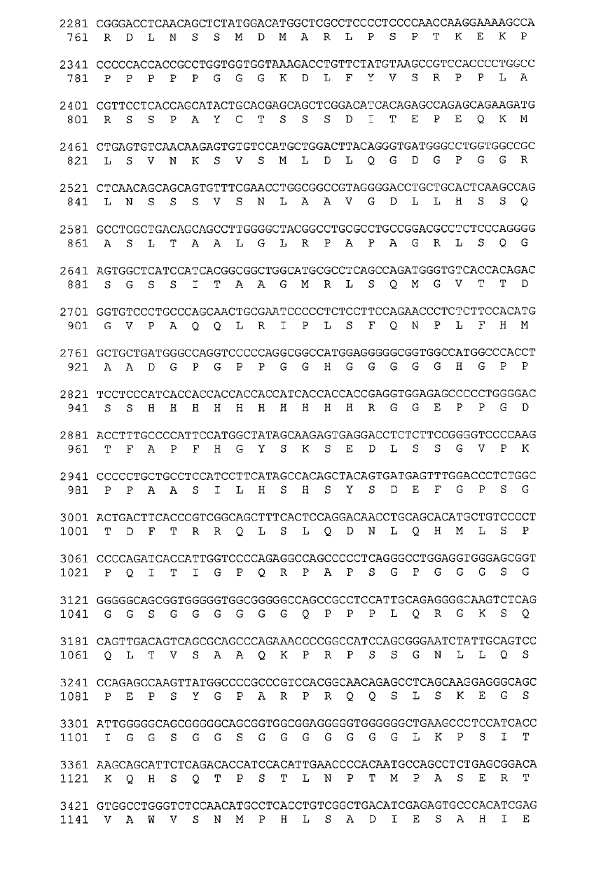

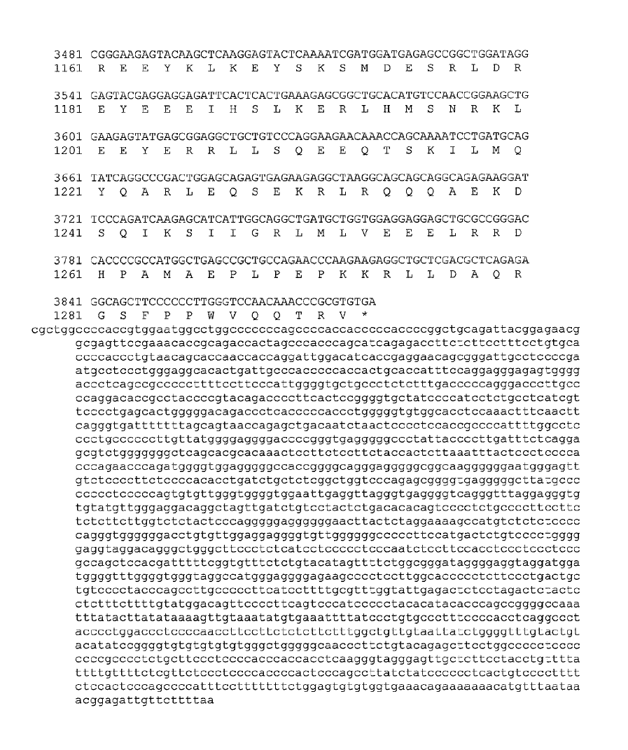

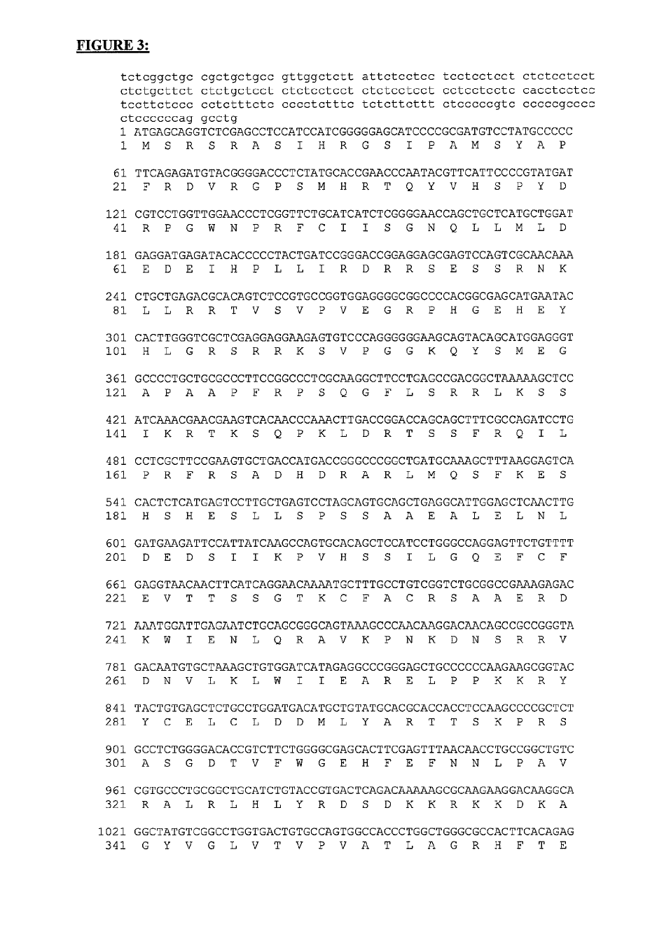

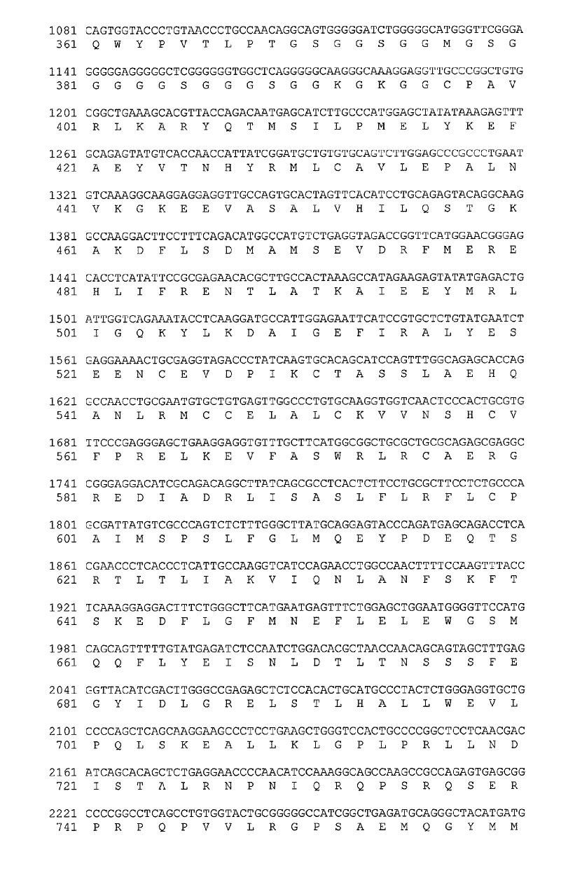

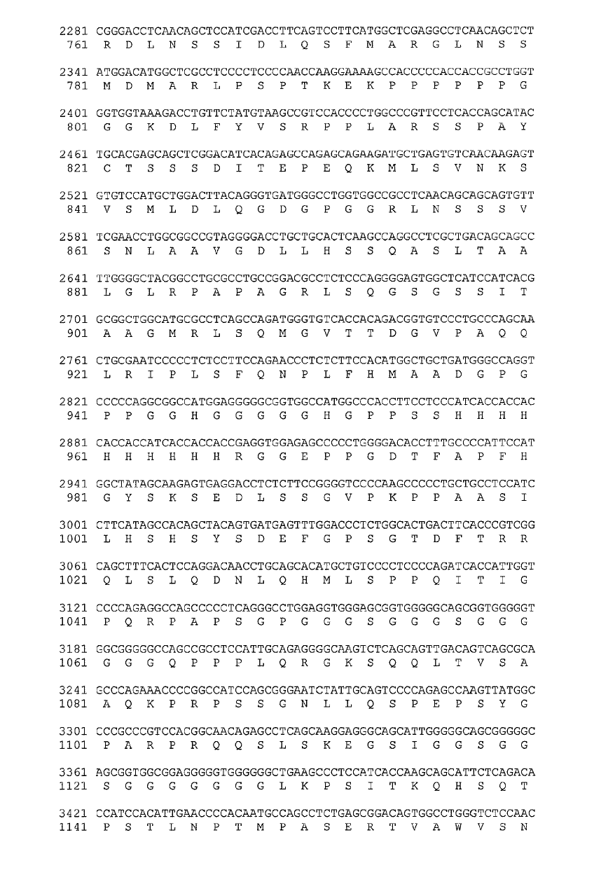

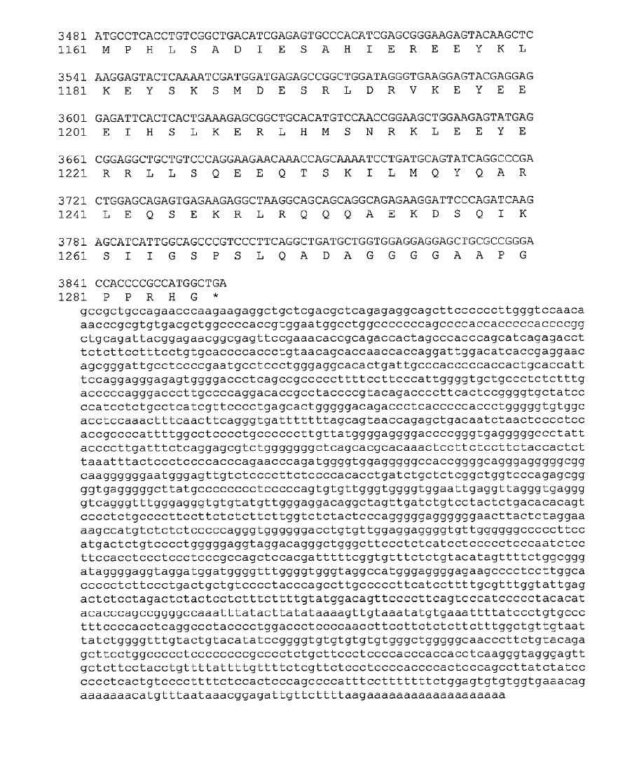

So far, at least three isoforms of the gene (i.e. isoforms 1, 2 and 3) have been detected in humans. The cDNA sequence of isoform 1 is shown in FIG. 1 and represented as SEQ ID NO:1 and is cited under NCBI Refseq #NM_{—}006772.2. Based on mRNA sequence information available from the rat Syngap1 (Li et al. 2003 JBC, 276: 21417-21424) showing extensive c-terminal splicing and other incomplete mRNA human SYNGAP1 sequences, at least 2 additional coding SYNGAP1 mRNAs, with different c-terminal coding sequences, could be also predicted in humans. Isoforms 2 and 3 are shown in FIGS. 2 and 3 and represented as SEQ ID NO:3 and SEQ ID NO:5 respectively. SYNGAP1 isoform 2 mRNA and corresponding protein sequences was predicted based on the c-terminal human mRNA sequence accession #AK307888, and is cited under NCBI Refseq#NM_{—}001130066. SYNGAP1 isoform 3 mRNA and corresponding protein sequences are based on the incomplete c-terminal human mRNA sequence accession #AL713634.

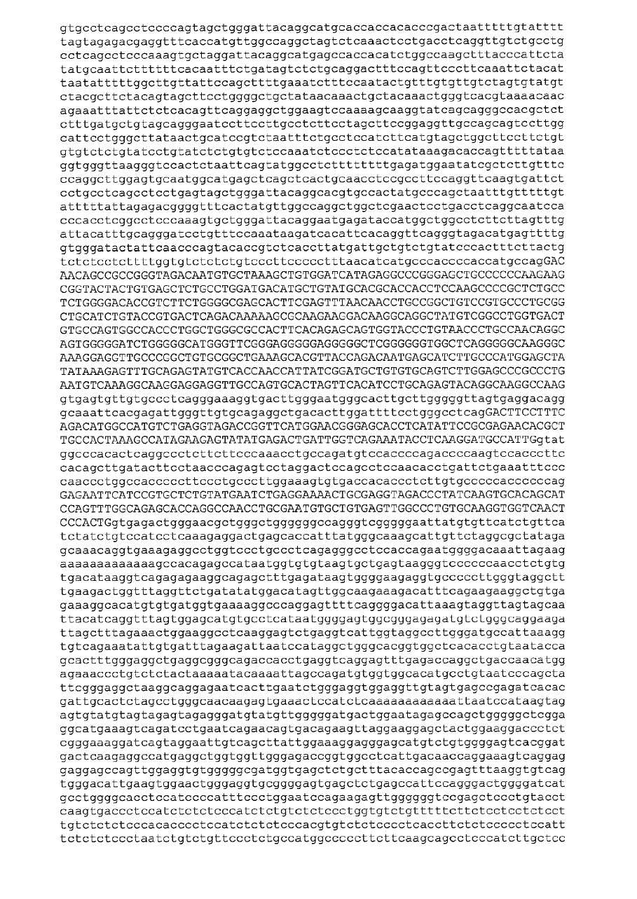

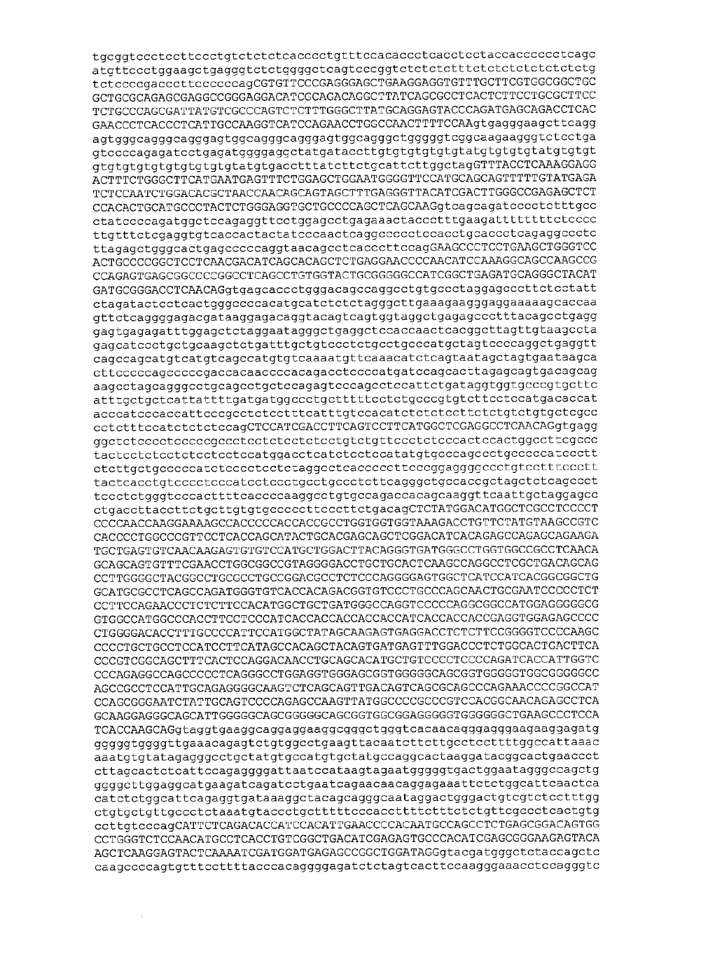

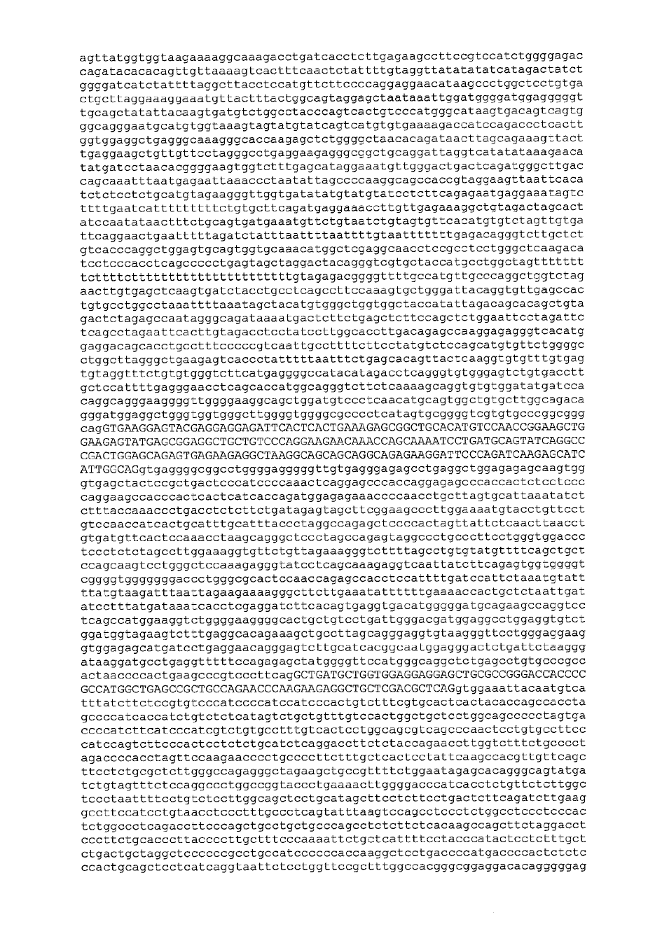

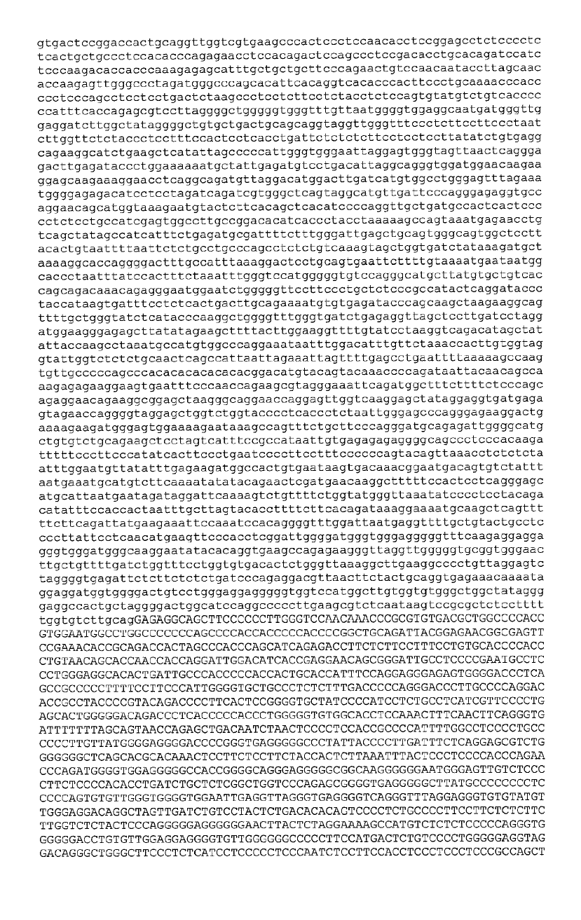

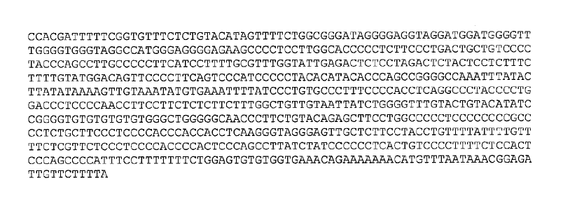

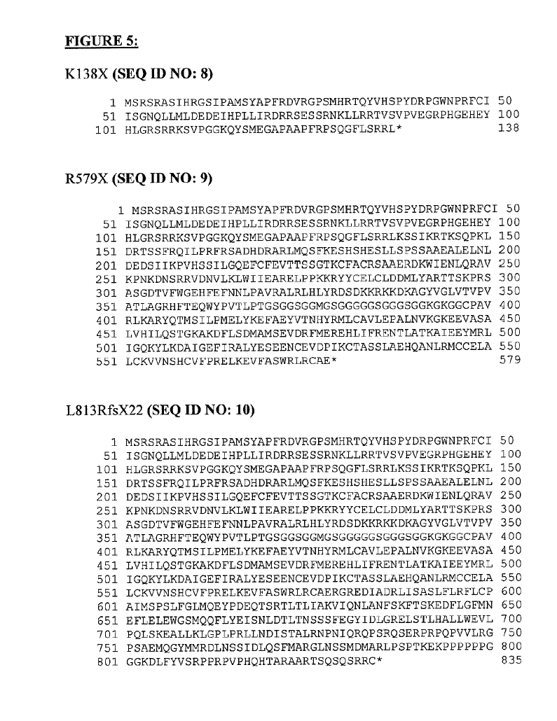

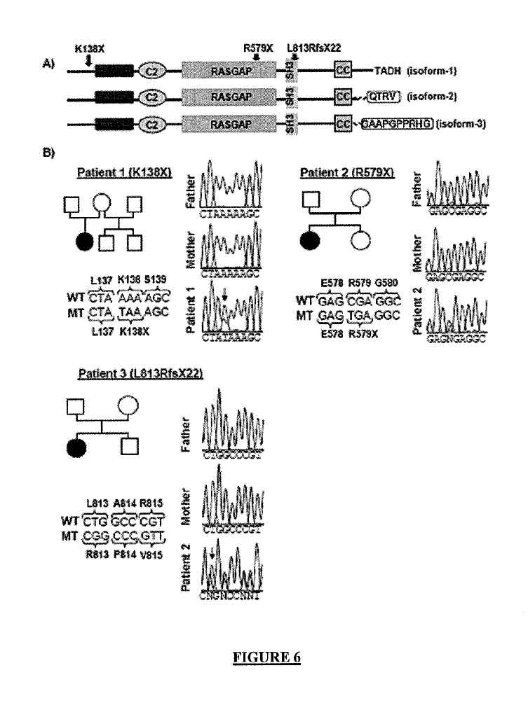

Syngap1 consists of 19 exons present in the 33.620 kb region on chromosome 6p21.32 with the following genomic position based on the NCBI hg18 assembly: chr6:33495825-33529444. Table 1 hereinafter lists the positions of the exons and introns in the genomic sequence for each of the three known/predicted isoforms. The amino acid sequences of isoform 1, isoform 2 and isoform 3 of the Syngap1 protein are shown in FIGS. 1, 2 and 3 and represented by SEQ ID NO:2, SEQ ID NO:4 and SEQ ID NO:6 respectively. FIG. 6 shows the position cDNA and amino acid positions (numbering based on isoform 1) of the de novo mutations identified in three young NSMR patients. FIG. 5 shows the predicted amino acid sequences (represented by SEQ ID NO:8, SEQ ID NO:8 and SEQ ID NO:10) of the truncated Syngap1 proteins found in the three NSMR patients.

TABLE 1
Exons and Introns positions for various SYNGAP1 isoforms*.

| Exon position | isoform 1 | isoform 2 | isoform 3 |
|---|---|---|---|
| exon 1; | 1-262; | 1-262; | 1-262; |
| (cds start) | (196) | (196) | (196) |
| exon 2 | 3408-3529 | 3408-3529 | 3408-3529 |
| exon 3 | 5729-5834 | 5729-5834 | 5729-5834 |
| exon 4 | 12092-12183 | 12092-12183 | 12092-12183 |
| exon 5 | 12616-12737 | 12616-12737 | 12616-12737 |
| exon 6 | 15083-15236 | 15083-15236 | 15083-15236 |
| exon 7 | 15446-15544 | 15446-15544 | 15446-15544 |
| exon 8 | 17599-18222 | 17599-18222 | 17599-18222 |
| exon 9 | 18350-18494 | 18350-18494 | 18350-18494 |
| exon 10 | 18706-18850 | 18706-18850 | 18706-18850 |
| exon 11 | 20660-20896 | 20660-20896 | 20660-20896 |
| exon 12 | 21104-21305 | 21104-21305 | 21104-21305 |
| exon 13 | 21512-21690 | 21512-21690 | 21512-21690 |
| exon 14 | 22384-22425 | 22384-22425 | 22384-22425 |
| exon 15 | 22820-23891 | 22820-23891 | 22820-23891 |
| exon 16 | 24375-24548 | 24375-24548 | 24375-24548 |
| exon 17 | 26506-26717 | 26506-26717 | 26506-26717 |
| exon 18; | 27774-27864 | 27774-27863 | 27761-27864; |
| (cds end) | | | (27821) |
| exon 19; | 31691-33620; | 31691-33620; | 31691-33620 |
| (cds end) | (31834) | (31730) | |

*The nucleotide positions in this table are based on SYNGAP1 genomic sequence (SEQ ID 7; chr6: 33495825-33529444), where the beginning of this genomic sequence is considered 1 and the end is 33620. The positions of the start and the end of the coding sequence (cds) for each isoform are indicated in parenthesis. Possible genomic modifications that could lead to predicted isoforms 2 and 3 include, but are not limited to:
for isoform 2: exon 18 ends up at position 27863 instead of 27864 (“G” at the end of exon 18 becomes intronic in isoform 2);
for isoform 3: the 13 intronic bases at pos. 27761-27773 upstream of exon 18 are spliced out as part of exon 18 (i.e. they are not intronic anymore) due to possible activation of cryptic donor splice site (27759-27760).

Exemplary nucleotide sequences encoding Syngap1 include SEQ ID NO:1, SEQ ID NO:3 and SEQ ID NO:5 (mRNA); and SEQ ID NO:7 (gene). A Syngap1 nucleotide sequence may have 75%, 80%, 85%, 90%, 95%, 97%, 99% or more homology with any of SEQ ID NO:1, NO:3, NO:5, NO:7. In accordance with the present invention, nucleic acids having the appropriate level of sequence homology with a nucleic acid molecule encoding Syngap1 may be identified by using sequencing and/or hybridization and washing conditions of appropriate stringency.

Syngap1 encoding nucleic acid molecules of the invention include cDNA, genomic DNA, RNA, and fragments thereof which may be single- or double-stranded.

Thus, this invention provides oligonucleotides having sequences capable of hybridizing with at least one sequence of a nucleic acid molecule of the present invention. In some embodiments, the nucleic acid molecule of the invention is a probe. In some embodiments, the nucleic acid molecule of the invention is a primer (see for instance Table 2 which lists PCR primers targeting the 19 exons of SYNGAP1).

Also contemplated in the scope of the present invention are oligonucleotide probes which specifically hybridize with the nucleic acid molecules of the invention. In preferred embodiments, the probe specifically hybridizes with mutated Syngap1 nucleic acid molecules (e.g. a nucleic acid having a sequence encoding a mutated Syngap1 protein) while not hybridizing with the wild type or “normal” sequence under high or very high stringency conditions. The invention also encompasses nucleic acid probes hybridizing specifically to a complementary strand of the nucleic acid molecule having a sequence encoding a mutated Syngap1 protein. Primers capable of specifically amplifying Syngap1 encoding nucleic acids described herein are also contemplated herein. As mentioned previously, such oligonucleotides are useful as probes and primers for detecting, isolating or amplifying altered Syngap1 genes.

In some embodiments, nucleic acid molecule of the invention has (i) a sequence complementary to any of SEQ ID NO:1, NO:3, NO:5, NO:7. In some embodiments, nucleic acid molecule of the invention has (ii) a sequence which hybridizes under stringent conditions to at least 10, 15, 25, 50, 100, 250 or more contiguous nucleotides of any of SEQ ID NO:1, NO:3, NO:5, NO:7. Yet, in other embodiments the nucleic acid molecule of the invention is (iii) a fragment comprising at least 10, 15, 25, 50, 100, 250 or more contiguous nucleotides of any of SEQ ID NO:1, NO:3, NO:5, NO:7 or of the nucleic acid molecules (i) and (ii) identified hereinabove. In some embodiments, the nucleic acid molecule is a fragment comprising a Syngap1 dysfunction, preferably a pathogenic Syngap1 mutation associated with NSMR. In some embodiments, the nucleic acid molecule targets the 5′ regulatory region of the Syngap1 gene. The invention also encompasses nucleic acid molecules hybridizing specifically to a complementary strand of any of (i), (ii) or (iii).

Nucleic acid molecules encoding the Syngap1 proteins of the invention may be prepared by three general methods: (1) synthesis from appropriate nucleotide triphosphates, (2) isolation from biological sources, and (3) mutation of nucleic acid molecule encoding Syngap1 protein. These methods utilize protocols well known in the art. The availability of nucleotide sequence information, such as the sequences provided herein, enables preparation of an isolated nucleic acid molecule of the invention by oligonucleotide synthesis. Synthetic oligonucleotides may be DNA synthesizers or similar devices. The resultant construct may be purified according to methods known in the art, such as high performance liquid chromatography (HPLC). Long, double-stranded polynucleotides may be synthesized in stages, due to any size limitations inherent in the oligonucleotide synthetic methods.

Nucleic acid sequences encoding the Syngap1 proteins of the invention may be isolated from appropriate biological sources using methods known in the art. In one embodiment, a cDNA clone is isolated from a cDNA expression library of human origin. In an alternative embodiment, utilizing the sequence information provided by the cDNA sequence, human genomic clones encoding altered Syngap1 proteins may be isolated. Additionally, cDNA or genomic clones having homology with human and other known mammalian Syngap1 (e.g. mouse, rat, etc) may be isolated from other species using oligonucleotide probes corresponding to predetermined sequences within the human and mouse Syngap1 encoding nucleic acids.

Nucleic acids of the present invention may be maintained as DNA in any convenient vector. Accordingly, the invention encompasses vectors comprising a nucleic acid molecule of the invention. The invention also encompasses host cells transformed with such vectors and transgenic animals comprising such a nucleic acid molecule of the invention. Those cells and animals could serve as models of disease in order to study the mechanism of the function of the Syngap1 gene and also allow for the screening of therapeutics.

In preferred embodiments, the vector, host cell or transgenic animal comprises a nucleic acid molecule encoding a mutated Syngap1 protein (e.g. pathogenic mutation). Methods for producing host cells and transgenic animals are known. Host cells include, but are not limited to, embryonic stem cells and neuronal cell lines. Transgenic animals can be selected from farm animals (such as pigs, goats, sheep, cows, horses, rabbits, and the like), rodents (such as rats, guinea pigs, mice, and the like), non-human primates (such as baboon, monkeys, chimpanzees, and the like), and domestic animals (such as dogs, cats, and the like). A transgenic animal according to the invention is an animal having cells that contain a transgene which was introduced into the animal or an ancestor of the animal at a prenatal (embryonic) stage. Those cells and transgenic animals can be useful to study the pathophysiology of Syngap1 mental retardation and also to use for screening various nucleic acid-based, antibody-based, protein-based and pharmacologically-based treatments for MR, and more particularly NSMR.

It will be appreciated by persons skilled in the art that variants (e.g., allelic variants) of Syngap1 sequences exist in the human population, and must be taken into account when designing and/or utilizing oligonucleotides of the invention. Accordingly, it is within the scope of the present invention to encompass such variants, with respect to the Syngap1 sequences disclosed herein or the oligonucleotides targeted to specific locations on the respective genes or RNA transcripts. Accordingly, the term “natural allelic variants” is used herein to refer to various specific nucleotide sequences of the invention and variants thereof that would occur in a human population. The usage of different wobble codons and genetic polymorphisms which give rise to conservative or neutral amino acid substitutions in the encoded protein are examples of such variants. Such variants would not demonstrate altered Syngap1 activity or protein levels. Additionally, the term “substantially complementary” refers to oligonucleotide sequences that may not be perfectly matched to a target sequence, but such mismatches do not materially affect the ability of the oligonucleotide to hybridize with its target sequence under the conditions described.

### III. Proteins

The invention encompasses proteins, polypeptides, fragments and mutants of the nucleic acid molecule described herein. Exemplary Syngap1 proteins include those comprising SEQ ID NO:2, SEQ ID NO:4 and SEQ ID NO:6 (normal); and those comprising SEQ ID NO:8, SEQ ID NO:9 and SEQ ID NO:10 (mutated).

A Syngap1 polypeptide sequence may have 75%, 80%, 85%, 90%, 95%, 97%, 99% homology or more with any of SEQ ID NO:2, NO:4, NO:6, SEQ ID NO:8, SEQ ID NO:9 and SEQ ID NO:10. A Syngap1 polypeptide sequence according to the invention may also comprise at least 10, 15, 25, 50, 100, 250 or more contiguous amino acids of any of SEQ ID NO:2, NO:4, NO:6, NO:9, NO:10.

In some embodiments, the Syngap1 polypeptide is an isolated mutated Syngap1 protein. In some embodiments, the Syngap1 polypeptide comprises a Syngap1 dysfunction, preferably a pathogenic Syngap mutation associated with NSMR.

Syngap1 proteins or polypeptides of the present invention may be prepared in a variety of ways, according to known methods. The proteins may be purified from appropriate sources, e.g., transformed bacterial or animal cultured cells or tissues, by immunoaffinity purification. The availability of nucleic acid molecules encoding Syngap1 protein enables production of the protein using in vitro expression methods and cell-free expression systems known in the art. In vitro transcription and translation systems are commercially available, e.g., from Promega Biotech (Madison, Wis.) or Gibco-BRL (Gaithersburg, Md.).

Alternatively, larger quantities of Syngap1 proteins or polypeptides may be produced by expression in a suitable prokaryotic or eukaryotic system. For example, part or all of a DNA molecule encoding for Syngap1 may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

Syngap1 proteins or polypeptides produced by gene expression in a recombinant prokaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

Syngap1 proteins or polypeptides of the invention, prepared by the aforementioned methods, may be analyzed according to standard procedures. For example, such proteins may be subjected to amino acid sequence analysis, according to known methods.

The present invention also provides antibodies capable of immunospecifically binding to proteins and polypeptides of the invention. Such antibodies may include, but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Such antibodies may be may be utilized, for example, in detection, as part of disease treatment methods, and/or may be used as part of diagnostic techniques.

Polyclonal antibodies directed toward Syngap1 protein, mutants and fragments thereof may be prepared according to standard methods. In a preferred embodiment, monoclonal antibodies are prepared, which react immunospecifically with the various epitopes of the Syngap1 protein. In preferred embodiments, the antibodies are immunogically specific mutated Syngap1 proteins and polypeptides. Monoclonal antibodies may be prepared according to general methods known in the art. Polyclonal or monoclonal antibodies that immunospecifically interact with wild-type and/or mutant Syngap1 proteins can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

In a preferred embodiment, an antibody according to the invention binds specifically to a mutated Synpap1 protein or fragment thereof (e.g. a truncated Syngap1 protein). More preferably, an antibody according to the invention binds with specificity to a truncated Syngap1 protein comprising an amino acid sequence selected from the group consisting of SEQ ID NO:2, SEQ ID NO:4 or SEQ ID NO:6; but do not bind to a non-truncated Syngap1 protein comprising an amino acid sequence according to SEQ ID NO:8, SEQ ID NO:9 and SEQ ID NO:10.

### IV. Detection Methods

Some aspects of the invention relate to methods for detecting a Syngap1 mutation, methods of detecting mental retardation in a human subject, methods of detecting non-syndromic mental retardation (NSMR) in a human subject. The methods of the invention are particularly useful for detecting de novo mutations (i.e. a mutation that is not found in the parents of an affected individual). The regions which may be targeted for detecting such a mutation includes the 5′ regulatory region of the Syngap1 gene, introns of Syngap1 gene, exons of the Syngap1 gene, or mRNAs of the Syngap1 gene.

There are numerous methods for detecting a mutation in a gene (see, in general, Ausubel et al. (1998) Current Protocols in Molecular Biology, John Wiley & Sons, New York. Exemplary approaches for detecting alterations in Syngap1 encoding nucleic acids include, without limitation:
a) sequencing regions of the DNA encoding a Syngap1 protein;
b) analyzing the sequence of nucleic acid molecules in a sample from a human subject for the detection of sequence abnormalities or dysfunctions (e.g. altering mutation, point mutation, truncation mutation, deletion mutation, frame-shift mutation, null mutation, splicing mutations, etc.);
c) comparing the sequence of nucleic acid molecules in a sample from a human subject with the wild-type Syngap1 nucleic acid sequence to determine whether the sample from the subject contains pathogenic mutations (e.g. altering mutation, point mutation, truncation mutation, deletion mutation, frame-shift mutation, and null mutation, nonsense mutation, missense mutation, mutation affecting exon splicing (consensus splice sites), etc.);
d) determining the presence, in a sample from a human subject, of the polypeptide encoded by the Syngap1 gene and, if present, determining whether the polypeptide is mutated, whether it is active (e.g. level of activity) and/or whether is expressed at a normal level;
e) using DNA restriction mapping to compare the restriction pattern produced when a restriction enzyme cuts a sample of nucleic acid from the subject with the restriction pattern obtained from normal Syngap1 gene or from known mutations thereof;
f) using a specific binding member capable of binding to a Syngap1 nucleic acid sequence (either normal sequence or known mutated sequence), the specific binding member comprising either nucleic acid molecules hybridizable with the Syngap1 sequence or substances comprising an antibody domain with specificity for Syngap1 nucleic acid sequence (either normal sequence or known mutated sequence) or the polypeptide encoded by it, the specific binding member being labeled so that binding of the specific binding member to its binding partner is detectable;
g) evaluating the number of copies of the Syngap1 gene using techniques such as array genomic hybridization, quantitative polymerase chain reaction (QPCR) or fluorescent in situ hybridization (FISH) on chromosomal preparations, or multiplex ligation dependent probe amplification (MLPA); and
h) using PCR involving one or more primers based on normal or mutated Syngap1 gene sequence to screen for normal or mutant Syngap1 gene in a sample from a human subject.

In one particular embodiment, a biological sample having DNA (e,g, genomic DNA) is obtained from the subject, the one or more regions of the DNA encoding the Syngap1 protein are sequenced and the sequenced region(s) is compared with a corresponding sequence from an unaffected individual. Identification of one or more Syngap1 mutation known to be pathogenic is correlated with MR, and more particularly with NSMR. In some embodiments, the presence of one or more Syngap1 mutation is also tested in both parents to determine if they also carry it. Presence of the mutation in an unaffected parent (“healthy” with no mental retardation or cognitive dysfunction) is suggestive that the observed mutation is unlikely to be causative of the disease. However, if the mutation is de novo (not transmitted from any of the parents) and is predicted to affect protein function (e.g., missense, nonsense, frameshifts, insertions and deletions) or mRNA processing and stability (splicing and regulatory element mutations), then this mutation is correlated with mental retardation. The invention however is not limited to de novo mutations only because pathogenic mutations in SYNGAP1 may also be inherited. These mutations could be inherited from one of the parents having a mild form of mental retardation.

Direct DNA sequencing can be carried out using Sanger sequencing methods where SYNGAP1 is targeted alone or with few other genes. Alternatively, it is conceivable to use massively parallel sequencing technologies including “next generation sequencers” such as Roche 454™, Illumina GAII™, Helicose tSMS™, and ABI SOLID™ which allows the sequencing of large DNA regions or even the whole genome. The presence or absence of a pathogenic Syngap1 dysfunction may be also be possible via a genotyping approach using any form of high density arrays.

A determination for the presence or absence of a pathogenic Syngap1 dysfunction is also possible at the mRNA level, for instance by sequencing complementary DNA (cDNA) for SYNGAP1 mutations. This approach could be applied in tissues expressing SYNGAP1 mRNA. In this scenario, mRNA is isolated and Reverse Transcribed to complementary DNA (cDNA) and then subjected to PCR (RT-PCR) using oligonucleotides targeting the complete coding sequence of SYNGAP1 isoforms. Resulting SYNGAP1 cDNA is then sequenced using DNA sequencing technologies.

Measuring the level and/or activity of Syngap1 may be carried out by measuring directly such Syngap1 level or activity, or by measuring a known surrogate marker (e.g. RAS, RAP). Methods for measuring Syngap1 activity depend on the quantification of its RASGAP and/or RAPGAP activity, as previously described (Chen et al., 1998 Neuron 20:895-904; Kim et al., 1998 Neuron 20: 683-691; Krapivinsky et al. 2004 Neuron 43:563-574). Furthermore, alternative techniques are conceivable at the protein level using for instance antibodies against SYNGAP1 (available commercially) to quantify protein expression levels from tissue samples that may express SYNGAP1. Although SYNGAP1 is mainly expressed in brain neurons; however, emerging technologies such as iPS (induced pluripotent stem cell) could be applied on non-neuronal cells readily obtained from the patient (e.g. from the skin) and induce the transformation differentiation into neuronal cells that could then express SYNGAP1. Having such cells would be one possibility for the direct detection and quantification of SYNGAP1 protein levels (e.g. by western blotting or ELISA). Similarly, SYNGAP1 mRNA from these neurons could be quantified using qPCR techniques.

More specific examples of detection methods are provided in the Exemplification section and herein below. In certain embodiments for detecting for mutant Syngap1 encoding nucleic acid molecules, the Syngap1 nucleic acid in the sample will initially be amplified, e.g. using PCR, to increase the amount of Syngap1 nucleic acid molecules as compared to other sequences present in the sample. This allows the target Syngap1 sequences to be detected with a high degree of sensitivity if they are present in the sample. This initial step may be avoided by using highly sensitive array techniques.

Hitherto uncharacterized variations in the Syngap1 gene can be identified and localized to specific nucleotides by comparison of nucleic acids from an individual with mental retardation with an unaffected individual, ideally his/her parents. Various screening methods are suitable for this comparison including, but not limited to, direct DNA sequencing, single strand conformation polymorphism analysis (SSCP), conformation shift gel electrophoresis (CSGE), heteroduplex analysis (HA), chemical cleavage of mismatched sequences (CCMS), denaturing gradient gel electrophoresis (DGGE), temperature gradient gel electrophoresis (TGGE), denaturing high performance liquid chromatography (dHPLC), ribonuclease cleavage, carbodiimide modification, and microarray analysis. See, e.g., Cotton (1993) Mutation Res. 285:125-144. Comparison can be initiated at either cDNA or genomic level. Initial comparison is often easier at the cDNA level because of its shorter size. Corresponding genomic changes are then identified by amplifying and sequencing a segment from the genomic exon including the site of change in the cDNA. In some instances, there is a simple relationship between genomic and cDNA changes. That is, a single base change in a coding region of genomic DNA gives rise to a corresponding changed codon in the cDNA. In other instances, the relationship between genomic and cDNA changes is more complex. Thus, for example, a single base change in genomic DNA creating an aberrant splice site can give rise to deletion of a substantial segment of cDNA.

The preceding methods may serve to identify particular genetic changes responsible for mental retardation. Once a change has been identified, individuals can be tested for that change by various methods. These methods include direct sequencing, allele-specific oligonucleotide hybridization, allele-specific amplification, ligation, primer extension and artificial introduction of extension sites (see Cotton, supra). Of course, the methods noted above for analyzing uncharacterized variations can also be used for detecting characterized variations. Certain methods are described in more detail below.

Mutational Analysis/Conformation Sensitive Gel Electrophoresis (CSGE). Conformation sensitive gel electrophoresis (CSGE) can be performed using standard protocols (Ganguly, A. et al. (1993) PNAS 90:10325-10329). PCR products corresponding to all altered migration patterns (shifts) can be purified and sequenced.

Isolation and Amplification of DNA. Samples of patient genomic DNA can be isolated from any suitable cell, fluid, or tissue sample. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W. H. Freeman & Co. New York (1992). Genomic DNA can also be isolated from cultured primary or secondary cell cultures or from transformed cell lines derived from any of the aforementioned tissue samples.

Samples of a human subject's RNA can also be used. RNA can be isolated from tissues expressing the Syngap1 gene as described in Sambrook et al., supra. RNA can be total cellular RNA, mRNA, poly A+ RNA, or any combination thereof. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al. (1990) Hum. Genet. 85:655-658.

PCR Amplification. The most common means for amplification is polymerase chain reaction (PCR), as described in U.S. Pat. Nos. 4,683,195, 4,683,202, and 4,965,188. To amplify a target nucleic acid sequence in a sample by PCR, the sequence must be accessible to the components of the amplification system. Methods of isolating target DNA by crude or fine extraction are known in the art. See, e.g., Higuchi, “Simple and Rapid Preparation of Samples for PCR”, in PCR Technology, Ehrlich, H. A. (ed.), Stockton Press, New York, and Miller et al. (1988) Nucleic Acids Res. 16:1215. Notably, kits for the extraction of DNA for PCR are also readily available.

Allele Specific PCR. Allele-specific PCR differentiates between target regions differing in the presence or absence of a mutation. PCR amplification primers are chosen which bind only to certain alleles of the target sequence, e.g., a Syngap1 gene comprising a mutation. Thus, for example, amplification products are generated from those samples which contain the primer binding sequence and no amplification products are generated in samples without the primer binding sequence. This method is described by Gibbs (1989) Nucleic Acid Res. 17:12427-2448. Allele Specific

Oligonucleotide Screening Methods. Further diagnostic screening methods employ the allele-specific oligonucleotide (ASO) screening methods, as described by Saiki et al. (1986) Nature 324:163-166. Oligonucleotides with one or more base pair mismatches are generated for any particular Syngap1. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed so that under low stringency, they will bind to both wild-type and mutant forms of Syngap1, but at higher stringency, they will bind to the form to which they correspond. Alternatively, stringency conditions can be devised in which an essentially binary response is obtained, i.e., an ASO corresponding to a mutant form of the Syngap1 gene will hybridize to that allele and not to wild-type Syngap1.

Ligase Mediated Allele Detection Method. Target regions of a human subject can be compared with target regions in unaffected individuals by ligase-mediated allele detection. See, e.g., Landegren et al. (1988) Science 241:1077-1080. Ligase may also be used to detect point mutations in the ligation amplification reaction described in Wu et al. (1989) Genomics 4:560-569. The ligation amplification reaction (LAR) utilizes amplification of specific DNA sequence using sequential rounds of template dependent ligation as described in Wu et al. and Barany (1990) PNAS 88:189-193.

Denaturing Gradient Gel Electrophoresis. Amplification products generated using the polymerase chain reaction can be analyzed by the use of denaturing gradient gel electrophoresis. Different mutations/alleles can be identified based on the different sequence-dependent melting properties and electrophoretic migration of DNA in solution.

Differentiation between mutant and wild-type sequences based on specific melting domain differences can be assessed using polyacrylamide gel electrophoresis, as described, for example, in Chapter 7 of Erlich, ed., PCR Technology, Principles and Applications for DNA Amplification, W. H. Freeman and Co, New York (1992).

Generally, a target region to be analyzed by denaturing gradient gel electrophoresis is amplified using PCR primers flanking the target region. The amplified PCR product is applied to a polyacrylamide gel with a linear denaturing gradient as described, for example, in Myers et al. (1986) Meth. Enzymol. 155:501-527 and Myers et al., in Genomic Analysis, A Practical Approach, K. Davies Ed. IRL Press Limited, Oxford, pp. 95-139 (1988). The electrophoresis system is maintained at a temperature slightly below the Tₘ of the melting domains of the target sequences.

In an alternative method of denaturing gradient gel electrophoresis, the target sequences may be initially attached to a stretch of GC nucleotides, termed a GC clamp, as described, for example, in Chapter 7 of Erlich, supra. Preferably, at least 80% of the nucleotides in the GC clamp are either guanine or cytosine. Preferably, the GC clamp is at least 30 bases long. This method is particularly suited to target sequences with high melting temperatures.

Gradient Gel Electrophoresis. Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel, the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis. Target sequences or mutants at the Syngap1 locus can be differentiated using single-strand conformation polymorphism analysis, which identifies base differences by alteration in electrophoretic migration of single stranded PCR products, as described, for example, in Orita et al. (1989) PNAS 86:2766-2770. Amplified PCR products can be generated as described above, and heated or otherwise denatured, to form single-stranded amplification products. Single-stranded nucleic acids may refold or form secondary structures which are partially dependent on the base sequence. Thus, electrophoretic mobility of single-stranded amplification products can detect base-sequence difference between alleles or target sequences. Chemical or Enzymatic Cleavage of Mismatches Differences between target sequences can also be detected by differential chemical cleavage of mismatched base pairs, as described, for example, in Grompe et al. (1991) Am. J. Hum. Genet. 48:212-222. In another method, differences between target sequences can be detected by enzymatic cleavage of mismatched base pairs, as described, for example, in Nelson et al. (1993) Nature Genetics 4:11-18. Briefly, genetic material from a human subject and an unaffected individual may be used to generate mismatch free heterohybrid DNA duplexes. As used herein, “heterohybrid” means a DNA duplex strand comprising one strand of DNA from one person, usually the subject, and a second DNA strand from another person, usually an unaffected individual. Positive selection for heterohybrids free of mismatches allows determination of small insertions, deletions or other polymorphisms that may be associated with mental retardation.

Non-PCR Based DNA Diagnostics. The identification of a DNA sequence linked to Syngap1 can made without an amplification step, based on polymorphisms including restriction fragment length polymorphisms in a human subject and a normal individual. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which may be bound to avidin or streptavidin, or peptide or protein tags. Visual detection methods include, without limitation, photoluminescents, chemoluminescence, horse radish peroxidase, alkaline phosphatase, and the like.

### V. Screening Methods

With the identification and sequencing of pathogenic Syngap1 dysfunctions and mutated Syngap1 proteins, it is now possible to use nucleic acid probes and specific antibodies in a variety of hybridization and immunological assays to screen for and detect the presence of either a normal or a mutated Syngap1 gene or gene product in a subject such as a human. Assays may in general also be used to detect the activity of the Syngap1 proteins. The invention thus encompasses assay kits and methods for such screening of possible therapeutic compounds and compositions to help alleviate, treat and/or prevent the disease.

According to another aspect of the invention, methods of screening drugs to identify suitable drugs for restoring Syngap1 function(s) are provided. One technique for drug screening involves the use of host eukaryotic cell lines, animals (e.g. transgenic animal) or cells which have a mutant Syngap1 gene. These host cell lines, animals or cells are defective at the Syngap1 polypeptide level. The host cell lines, or animal or cells are placed in the presence of a test compound. The restoration of Syngap1 activity or increased Syngap1 protein levels, for example, in the presence of the test compound suggests the compound is capable of restoring Syngap1 function(s) to the cells.

Based on the biochemical analyses of Syngap1 protein structure-function, one can design drugs to mimic the effects of Syngap1 on target proteins. Recombinant Syngap1 expressed as a fusion protein can be utilized to identify small peptides that bind to Syngap1 such as by using a phage display approach. An alternate but related approach uses the yeast two-hybrid system to identify further binding partners for Syngap1.

### VI. Kits

A further aspect of the invention relates to a solid support and to kits. The solid supports and/or kits of the invention may be useful for the practice of the methods of the invention, particularly for diagnostic applications in humans according to the evaluation methods described hereinbefore.

A solid support the invention may comprise a compound for identifying a pathogenic Syngap1 dysfunction in a human subject, wherein the dysfunction is responsible for mental retardation. In one embodiment, the compound is a nucleic acid probe designed for specific detection of a Syngap mutation associated with non-syndromic mental retardation (NSMR). The solid support may me a tube, a chip (see for instance Affimetrix GeneChip® technology), a membrane, a glass support, a filter, a tissue culture dish, a polymeric material, a bead, a silica support, etc.

A kit of the invention may comprise one or more of the following elements: a buffer for the homogenization of the biological sample(s), purified Syngap1 proteins (and/or a fragment thereof) to be used as controls, incubation buffer(s), substrate and assay buffer(s), modulator buffer(s) and modulators (e.g. enhancers, inhibitors), standards, detection materials (e.g. antibodies, fluorescein-labelled derivatives, luminogenic substrates, detection solutions, scintillation counting fluid, etc.), laboratory supplies (e.g. desalting column, reaction tubes or microplates (e.g. 96- or 384-well plates), a user manual or instructions, etc. Preferably, the kit and methods of the invention are configured such as to permit a quantitative detection or measurement of the protein(s) or nucleotide of interest.

For instance, the kits may comprise at least one oligonucleotide which specifically hybridizes with mutant Syngap1 encoding nucleic acid molecules, reaction buffers, and instructional material. Optionally, the at least one oligonucleotide contains a detectable tag. Certain kits may contain two such oligonucleotides, which serve as primers to amplify at least part of the Syngap1 gene. The part selected for amplification can be a region from the Syngap1 gene that includes a site at which a mutation is known to occur. Some kits contain a pair of oligonucleotides for detecting pre-characterized mutations. Alternatively, the kit may comprise primers for amplifying at least part of the Syngap1 gene to allow for sequencing and identification of mutant Syngap1 nucleic acid molecules. The kits of the invention may also contain components of the amplification system, including PCR reaction materials such as buffers and a thermostable polymerase. In other embodiments, the kit of the present invention can be used in conjunction with commercially available amplification kits, such as may be obtained from GIBCO BRL (Gaithersburg, Md.) Stratagene (La Jolla, Calif.), Invitrogen (San Diego, Calif.). The kits may optionally include instructional material, positive or negative control reactions, templates, or markers, molecular weight size markers for gel electrophoresis, and the like.

Kits of the instant invention may also comprise antibodies immunologically specific for Syngap1 protein(s) and/or mutants thereof and instructional material. Optionally, the antibody contains a detectable tag. The kits may optionally include buffers for forming the immunocomplexes, agents for detecting the immunocomplexes, instructional material, solid supports, positive or negative control samples, molecular weight size markers for gel electrophoresis, and the like.

### V. Therapeutics

The discovery that mutations in the Syngap1 gene give rise to mental retardation facilitates the development of pharmaceutical compositions useful for treatment and diagnosis of this syndrome and condition.

SYNGAP1 is a neuron-specific GTPase activating protein (GAP) that inhibits the activity of the small GTPases RAS and RAP (Chen et al., 1998 Neuron 20:895-904; Kim et al., 1998 Neuron 20: 683-691; Pena et al. 2008 EMBO Rep 9:350-5.). RAS and RAP are important for signalling of the α-amino-3-hydroxy-5-methylisoxazole-4-propionic acid (AMPA) glutamate receptors (AMPAR) during long-term synaptic potentiation (LTP) and depression (LTD), respectively (Zhu et al. 2002 Cell 110:443-55). SYNGAP1 is selectively expressed in excitatory synapses where it associates with the NR2B subunit of the N-methyl-D-asparate (NMDA) receptors as well as synaptic adaptor and signalling proteins such as PSD95, SAP102, MUPP1, and Ca++/calmodulin-dependent kinase (CamKII) (Chen et al., 1998 Neuron 20:895-904; Kim et al., 1998 Neuron 20: 683-691; Krapivinsky et al. 2004 Neuron 43:563-574). Nearly all presynaptic terminals that make synapses on dendritic spines release the neurotransmitter glutamate. Glutamate signalling via NMDAR located at the surface of spines is necessary for the plasticity of excitatory synapses. The NMDAR is linked to multiple pathways through its association with a large complex of more than 185 proteins (Laumonnier et al. 2007 Am J Hum Genet 80:205-220). Some forms of cognition and synaptic plasticity that are regulated by NMDAR require the insertion of AMPAR at the post-synaptic membrane (Shepherd and Huganir 2007 Annu Rev Cell Dev Biol 23:613-643). SYNGAP1 has been shown to act downstream of NMDAR to regulate AMPAR trafficking insertion at the post-synaptic membrane through a mechanism involving, the inhibition of members of the Ras-ERK-MAPK pathway (Krapivinsky et al. 2004 Neuron 43:563-574; Kim et al., 2005 Neuron 46:745-60; Rumbaugh et al., 2006 PNAS 103:4344-4351). Over expression of mouse Syngap1 in neurons results in decrease of AMPAR-mediated synaptic transmission, a significant reduction in synaptic AMPAR surface expression, and a decrease in the synaptic AMPARs surface expression; in contrast, synaptic transmission is augmented in neurons from SYNGAP1 knockout mice as well as in neuronal cultures treated with SYNGAP1 small interfering RNA (Rumbaugh et al., 2006 PNAS 103:4344-4351). Mice homozygous for null alleles of Syngap1 die shortly after birth, indicating an essential role for Syngap1 during early postnatal development, while Syngap1 heterozygous mice display phenotypes of impaired synaptic plasticity and learning, consistent with its function in the NMDAR complex (Komiyama et al. 2002 J Neurosci 22:9721-32; Kim et al., 2003 J Neurosci 23:1119-1124).

Because Syngap1 activity is primarily found in the synapses, preferred therapeutic compounds would be capable of crossing the blood brain barrier (BBB).

Among potentially useful compounds are compounds that modify the activity of ribosomes allowing translational read-through premature stop codons caused by nonsense mutations (Welch et al., 2007 Nature 447(7140):87-91). One such compound is PTC124 which is in clinical trials for Cystic fibrosis and Duchenne muscular dystrophy arising from non-sense mutations in the CFTR and DMD genes, respectively (Kerem et al. 2008 Lancet 372 (9640): 719-27)

Other potentially therapeutically useful drugs include inhibitors of RAS or RAP or effectors of these pathways.

The pharmaceutical compositions of the invention may comprise a therapeutic agent (e.g. an agent identified by the above screens or a nucleic acid molecule encoding for wild-type Syngap1) in a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, and intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The methods may also be used advantageously for in utero screening of fetuses for the presence of a mutant Syngap1. Identification of such variations offers the possibility of gene therapy. For couples known to be at risk of giving rise to affected progeny, diagnosis can be combined with in vitro reproduction procedures to identify an embryo having wild-type Syngap1 before implantation. Screening children shortly after birth is also of value in identifying those having a pathogenic Syngap1 dysfunction. Early detection allows administration of appropriate treatment.

As a further alternative, the nucleic acid encoding the wild-type Syngap1 polypeptide could be used in a method of gene therapy, to treat a human subject who is unable to synthesize the active protein to normal levels, thereby restoring normal Syngap1 function(s). For instance, patient therapy through supplementation with the normal gene product, whose production can be amplified using genetic and recombinant techniques, or its functional equivalent, is now conceivable. Correction or modification of the defective gene product through drug treatment means is embodied. In addition, NSMR may be treated or controlled through gene therapy by correcting the gene defect in situ or using recombinant or other vehicles to deliver a DNA sequence capable of expression of the normal gene product to the cells of the subject.

Vectors, such as viral vectors have been used in the prior art to introduce genes into a wide variety of different target cells. Typically, the vectors are exposed to the target cells so that transformation can take place in a sufficient proportion of the cells to provide a useful therapeutic or prophylactic effect from the expression of the desired polypeptide. The transfected nucleic acid may be permanently incorporated into the genome of each of the targeted cells, providing long lasting effect, or alternatively the treatment may have to be repeated periodically. A variety of vectors for gene therapy, both viral vectors and plasmid vectors, are known in the art.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further limiting.

### EXAMPLE

### Example 1

### De Novo Mutations in SYNGAP1, a Component of the NMDA Receptor Complex Cause Autosomal Non-Syndromic Mental Retardation

### Summary

Non-syndromic mental retardation (NSMR) represents one of the most important unsolved problems in medicine. Although autosomal forms of NSMR account for the majority of cases, the genes involved remain largely unknown. The autosomal gene SYNGAP1, which codes for a RAS GTPase-activating protein that is critical for cognition and synapse function, was sequenced in 94 patients with NSMR and de novo truncating mutations (K138X, R579X, L813RfsX22) were identified in three of them. In contrast, no SYNGAP1 de novo or truncating mutations were found in controls (n=190). SYNGAP1 is the first example of an autosomal dominant NSMR gene.

### Methods

Patients. A cohort of 94 sporadic cases of NSMR (45 males, 49 females) was recruited for this study. All patients were examined by at least one experienced clinical geneticist who ruled out the presence of specific dysmorphic features. Birth weight and postnatal growth were unremarkable. Head circumference was normal at birth for all patients. The diagnosis of MR was made on a clinical basis using standardized developmental or IQ tests. MR was unexplained in these cases despite standard investigations, including subtelomeric FISH studies, karyotyping, or CGH targeting regions associated with known syndromes, molecular testing for the common expansion mutation in FMR1, and brain CT-scan or MRI. Cohorts of 190 healthy ethnically-matched controls were also studied. Blood samples were obtained from all members of these cohorts as well as from their parents. Samples were collected through informed consent after approval of each of the studies by the respective institutional ethics committees. Genomic DNA was extracted from blood samples using the Puregene DNA kit and according to the manufacturer's protocol (Gentra System, USA). Paternity and maternity of each individual of all families were confirmed using 6 highly informative unlinked microsatellite markers (D2S1327, D3S1043, D4S3351, D6S1043, D8S1179, D10S677).

Gene screening, validation analyses and bioinformatics. SYNGAP1 (chr6:33495825-33529444; Refseq NM_{—}006772; NCBI Build 36.1) coding regions and their intronic flanking regions were amplified by PCR from genomic DNA and the resulting products were sequenced. PCR primers targeting all SYNGAP1 19 exons were designed using Exon-Primer from the UCSC Genome Browser (Table 2). PCR was done in 384 well plates using 5 ng of genomic DNA, according to standard procedures. PCR products were sequenced at the McGill University and Genome Quebec Innovation Centre (Montreal, Canada) (www.genomequebecplatforms.com/mcgill/) on a 3730XL™ DNA Analyzer. In each case, unique mutations were confirmed by re-amplifying the fragment and the re-sequencing of the proband and both parents using reverse and forward primers. PolyPHRED™ (v.5.04), PolySCAN™ (v.3.0) and Mutation Surveyor™ (v.3.10) were used for mutation detection analyses.

TABLE 2
Primer pairs used for PCR amplification of SYNGAP1 exons and
their intronic junctions

| | Amplicon | amplicon | | |
|---|---|---|---|---|
| Exon* | name | size (bp) | Forward Primer | Reverse Primer |
| 1 | G00223_054 | 355 | GGTCTCGAGCCTCCATCCATC | TTTTCCCCAACCCAATCCTTCTAC |
| | | | (SEQ ID NO: 11) | (SEQ ID NO: 12) |
| 2 | G00223_002 | 331 | CTTGCCATTTTAGGCCTCTG | AGTCTCAATGGCCACCCTC |
| | | | (SEQ ID NO: 13) | (SEQ ID NO: 14) |
| 3 | G00223_003 | 260 | CTTCCTGGGAGGAGGCG | CAGCCCGGTCCATCTTC |
| | | | (SEQ ID NO: 15) | (SEQ ID NO: 16) |
| 4 | G00223_004 | 245 | GGGAACCTGGGTTAACAGC | TCTTTCTCAGACTCCTAGGGC |
| | | | (SEQ ID NO: 17) | (SEQ ID NO: 17) |
| 5 | G00223_005 | 278 | ATCCAGGGGCTCTCTACCAG | CCCCTCCCTCTGCATCTC |
| | | | (SEQ ID NO: 19) | (SEQ ID NO: 20) |
| 6 | G00223_006 | 429 | AAGTTGCAGCAAGCCGAG | CCTACCCTTTCCTCCAGTCC |
| | | | (SEQ ID NO: 21) | (SEQ ID NO: 22) |
| 7 | G00223_007 | 252 | GGGAGGAAGAGAAGGTAGCAG | ACTTTCCTCCCTAGGCCCC |
| | | | (SEQ ID NO: 23) | (SEQ ID NO: 24) |
| 8.1 | G00223_059 | 367 | TTGCAGGGATCCTGTTTCC | TGCTCGCCCCAGAAGAC |
| | | | (SEQ ID NO: 25) | (SEQ ID NO: 26) |
| 8.2 | G00223_060 | 242 | TACTGTGAGCTCTGCCTGG | TGCTCTGTGAAGTGGCG |
| | | | (SEQ ID NO: 27) | (SEQ ID NO: 28) |
| 8.3 | G00223_009 | 450 | GAAGGACAAGGCAGGCTATG | GCCCTGTCCTCACTAACCC |
| | | | (SEQ ID NO: 29) | (SEQ ID NO: 30) |
| 9 | G00223_010 | 296 | AGTGAGGACAGGGCAAATTC | AAGCTGTGGAAGGGTGGAC |
| | | | (SEQ ID NO: 31) | (SEQ ID NO:32) |
| 10 | G00223_025 | 512 | CAGATGTCCACCCCAGACC | AATTTGTCCCCATTCTGGTG |
| | | | (SEQ ID NO: 33) | (SEQ ID NO: 34) |
| 11 | G00223_012 | 402 | CTGGAAGCTGAGGGTCTCTG | AGACCCTTCTTGCCGACC |
| | | | (SEQ ID NO: 35) | (SEQ ID NO: 36) |
| 12 | G00223_013 | 372 | GGGAGGCTATGATACCTTGTG | AGGGTAGTTTCTCAGGCTCC |
| | | | (SEQ ID NO: 37) | (SEQ ID NO: 38) |
| 13 | G00223_014 | 343 | CTATCCCAACTCAGGCCCC | GGGCCCAGTGAGGAGTATC |
| | | | (SEQ ID NO: 39) | (SEQ ID NO: 40) |
| 14 | G00223_015 | 200 | CCGCCTCTCCTTTCATTTG | AGAGGAGTAGGGCGAAGGC |
| | | | (SEQ ID NO: 41) | (SEQ ID NO: 42) |
| 15.1 | G00223_016 | 481 | CCAGACCACAGCAAGGTTC | TCTGTGGTGACACCCATCTG |
| | | | (SEQ ID NO: 43) | (SEQ ID NO: 44) |
| 15.2 | G00223_017 | 469 | CGCTGACAGCAGCCTTG | AGCATGTGCTGCAGGTTG |
| | | | (SEQ ID NO: 45) | (SEQ ID NO: 46) |
| 15.3 | G00223_032 | 698 | CCCCCTGCTGCCTCCATCCTTCAT | AAGCCCCCAGCTGGCCCTATTCC |
| | | | (SEQ ID NO: 47) | (SEQ ID NO: 48) |
| 16 | G00223_019 | 337 | GTCTCCTTTGGCTGTGCTG | GGAAGTGACTAGAGATCTCCCC |
| | | | (SEQ ID NO: 49) | (SEQ ID NO: 50) |
| 17 | G00223_020 | 379 | ACAGGGATGGAGGCTGG | TTTGGGGATGGGAGTCAG |
| | | | (SEQ ID NO: 51) | (SEQ ID NO: 52) |
| 18 | G00223_021 | 258 | TCCAGAGAGCTATGGGGTTC | GCTAGGTGGCTGGTGTAGTG |
| | | | (SEQ ID NO: 53) | (SEQ ID NO: 53) |
| 19 | G00223_022 | 316 | CTATAGGGGAGGCCACTGC | ATGTCCAATCCTGGTGGTTG |
| | | | (SEQ ID NO: 55) | (SEQ ID NO: 56) |

*Exons 8 and 15 were divided each into 3 overlapping amplicons.

### Results

The coding regions of all 19 SYNGAP1 exons and their flanking intronic regions was sequenced in the cohort of 94 sporadic cases of NSMR. Sporadic cases were selected to increase the likelihood of finding de novo mutations. This led to the identification of two patients who are heterozygous for the nonsense mutations K138X (patient 1) and R579X (patient 2). In addition, a third patient was identified, that patient being heterozygous for the mutation c.2438deIT (patient 3), which is predicted to cause a frameshift starting at codon 813, producing a premature stop codon at position 835 (L813RfsX22) (FIG. 6). These three mutations were not found in blood DNA of the parents of the affected individuals, indicating that they are de novo, nor were they present in a control cohort of 190 healthy individuals in which all SYNGAP1 exons and intronic junctions were sequenced. Only one heterozygous missense variant (11115T), that was also present in controls, was found in the remaining NSMR cohort (Table 3).

TABLE 3
SYNGAP1 amino acid altering mutations
found in NSMR and control cohorts.

| Cohort | Mutation | Δ amino acid | Occurrence | Inheritance |
|---|---|---|---|---|
| NSMR | c.412A > T | K138X | 1/94 | De novo |
| | c.1735C > T | R579X | 1/94 | De novo |
| | c.2438delT | L813RfsX22 | 1/94 | De novo |
| | c.3344T > C | I1115T | 2/94 | ND |
| Controls¹ | c.603T > G | D201E | 1/190 | Father¹ |
| | c.2246G > A | R749Q | 1/190 | Father¹ |
| | c.3344T > C | I1115T | 4/190 | ND |

¹healthy individuals.
All reported mutations are heterozygous.
ND, not determined.
Mutation positions are according to the coding sequence of SYNGAP1 Refseq no. NM_006772.
“c.” indicates coding sequence.

The three patients with the de novo mutations, whose ages range between 4 and 11 years, showed a similar clinical picture (Table 4). They were born to non-consanguineous parents after uneventful pregnancies and deliveries. Early development was characterized by global delay and hypotonia with onset of walking at age 2. Mullen Scales of Early Learning and the Vineland Adaptive Behavioural Scale showed profiles that are consistent with moderate to severe MR in all patients. Non-verbal social interactions were unremarkable. In particular, evaluation of patient 3 with the Autism Diagnostic Observation Schedule was negative. Ophthalmologic assessment revealed a strabismus in patient 1. Two of the patients were mildly epileptic. Patient 1 had brief generalized tonic-clonic seizures and is seizure-free on topiramate, whereas patient 2 displayed some myoclonic and absence seizures which are well controlled with valproate. In both cases, an electroencephalogram revealed bi-occipital spikes during intermittent light stimulation.

TABLE 4
Clinical features of patients with SYNGAP1 de novo mutations

| Patient # | 1 | 2 | 3 |
|---|---|---|---|
| De novo mutation | K138X | R579X | L813RfsX22 |
| Age | 4 yrs 5 mo | 5 yrs 10 mo | 12 yrs 2 mo |
| Gender | female | female | female |
| Ethnic origin | South American | French Canadian | French Canadian |
| Weight (kg/centile rank) | 21.9/95 | 18.0/50 | 39.1/25-50 |
| Height (cm/centile rank) | 104/50 | 108.7/50 | 141.5/10 |
| Head circumference(cm/centile rank) | 48.3/3-10 | 52/75 | 52/25 |
| Epilepsy | + | + | − |
| Mullen Scales of Early Learning | | | |
| (centile rank/age equivalent in months) | | | |
| fine motor skills | <1 (17 months) | <1 (27 months) | <1 (31 months) |
| visual reception | <1 (25 months) | <1 (27 months) | <1 (34 months) |
| receptive language | <1 (14 months) | <1 (28 months) | <1 (36 months) |
| expressive language | <1 (10 months) | <1 (26 months) | <1 (23 months) |
| Vineland Adaptive Behavioural | | | |
| Scale (centile rank) | | | |
| Communication | <1 | 1 | <1 |
| Daily living skills | <1 | 6 | <1 |
| Socializing | <1 | 2 | <1 |
| Motor skills | <1 | 1 | <1 |
| Adaptive Behaviour Composite | <1 | 1 | 1 |
| Brain imaging | | | |
| MRI | normal | normal | ND |
| CT-Scan | ND | ND | normal |

ND, not determined

The K138X mutation is predicted to truncate SYNGAP1 before important functional domains such as a pleckstrin homology domain (PH), which binds phospholipids and might act as membrane recruitment motifs, a C2 domain which is required for RAPGAP activity, a RASGAP domain, a proline rich region that may form binding sites for SH3 domains, and a coiled coil domain (CC) (Kim et al., 1998 Neuron 20,683-691; Pena et al., 2008 EMBO Rep 9,350-355) (FIG. 6). The R579X and c.2438deIT mutations are predicted to truncate SYNGAP1 in the middle and just after the RASGAP domain, respectively. These three mutations occur upstream of the carboxyl region of the gene that is subjected to alternative splicing, as described for the rat Syngap1 (Li et al., 2001 J Biol Chem 276,21417-21424) (FIG. 6). This splicing process has the potential of producing at least 3 isoforms, including carboxyl-tails that can bind to other components of the NMDAR complex such as PSD95 and DLG3 (via the PDZ-binding motif, QTRV; isoform 2) or CamKII (via GAAPGPPRHG, isoform 3) (Kim et al., 1998 Neuron 20,683-691; Li et al., 2001 J Biol Chem 276,21417-21424). For instance, deletion of the QTRV motif impairs SYNGAP1 ability to bind PSD95 and DLG3 as well as regulate dendritic spine formation (Kim et al., 1998 Neuron 20,683-691; Vazquez et al., 2004 J Neurosci 24,8862-8872). As indicated hereinbefore, SYNGAP1 cDNA sequences deposited in GenBank™ support the existence of three SYNGAP1 isoforms in humans. The three mutations described here would thus result in the production of proteins that lack carboxy-domains that are crucial for SYNGAP1 function (See FIG. 5 for the predicted sequences of the resulting mutated proteins). Table 5 summarizes the predicted functional effect of the mutations.

TABLE 5
Prediction of the functional effect of the missense mutations detected
in SYNGAP1 using the programs SIFT, PolyPhen, and SNAP.

| Δ amino | SIFT | PolyPhen | SNAP % |
|---|---|---|---|
| acid | score/prediction | score/prediction | accuracy/prediction |
| D201E | 1.00/Tolerated | 0.08/Benign | 92/Neutral |
| T790N | 0.49/Tolerated | 0.07/Benign | 69/Neutral |
| R749Q | 0.57/Tolerated | 1.36/Benign | 78/Neutral |
| I1115T | 0.59/Tolerated | 0.54/Benign | 60/Neutral |

Tolerated, benign, and neutral, indicate that the amino acid modification is unlikely to affect protein function.
SIFT: http://blocks.fhcrc.org/sift/SIFT.html
PolyPhen: http://genetics.bwh.harvard.edu/pph/
SNAP: http://cubic.bioc.columbia.edu/services/SNAP/

### Discussion

This study led to the identification of protein-truncating de novo mutations in the autosomal gene SYNGAP1 in approximately 3% of the NSMR cohort. These mutations are likely pathogenic for several reasons. First, they all result in the production of proteins that lack domains, such RASGAP and/or QTRV, shown to be important for synaptic plasticity and spine morphogenesis which are required for learning and memory. In addition, the resulting premature stop codons could also act at the level of mRNA to destabilise SYNGAP1 transcript through the nonsense-mediated mRNA decay mechanism (Khajavi et al., 2006 Eur J Hum Genet 14,1074-1081). Second, mice heterozygous for null alleles of Syngap1 display impaired synaptic plasticity and learning, suggesting that disruption of a single SYNGAP1 allele is, likewise, sufficient to cause cognitive dysfunction in humans (Komiyama et al., 2002 J Neurosci 22,9721-9732; Kim et al., 2003 J Neurosci 23,1119-1124). Third, extensive screening of 190 individuals without NSMR failed to identify any truncating, splicing or de novo amino acid altering variants in SYNGAP1, reinforcing the idea that disruption of this gene is specifically associated with NSMR.

SYNGAP1 interacts with the NR2B subunit of NMDAR and with the synaptic adaptor proteins PSD95 and DLG3 (Kim et al., 1998 Neuron 20,683-691; Kim et al., 2005 Neuron 46,745-760). Knockout of Dlg3 affects synaptic plasticity and cognition in a mechanism that implicates NMDAR signalling (Cuthbert et al., 2007 J Neurosci 27,2673-2682). Interestingly, DLG3 also interacts with NR2B and mutations in DLG3 have been recently reported to cause X-linked NSMR (Tarpey et al., 2004 Am J Hum Genet 75,318-324). Regulation of AMPAR trafficking represents a major postsynaptic mechanism for modulating synaptic plasticity and cognition (Shepherd and Huganir, 2007 Annu Rev Cell Dev Biol 23,613-643). SYNGAP1 and DLG3 affect differently AMPAR synaptic trafficking. While SYNGAP1 inhibits the surface insertion of the AMPAR subunit GluR1 in adult hippocampal synapses by down regulating RAS-ERK signalling (Kim et al., 2005,46,745-760; Rumbaugh et al., 2006,103,4344-4351), DLG3, in contrast, stimulates AMPAR trafficking, mainly in immature synapses (Kim et al., 2005 Neuron 46,745-760; Elias et al., 2006 Neuron 52,307-320). This may explain why, unlike the case of Dlg3, knockout of Syngap1 has been shown to cause a marked increase in AMPAR-mediated synaptic transmission, probably as a consequence of increased AMPAR surface expression (Rumbaugh et al., 2006 PNAS 103,4344-4351; Cuthbert et al., 2007 J Neurosci 27,2673-2682). Therefore, although SYNGAP1 and DLG3 physically interact, they may affect cognitive process through different mechanisms. The critical role of AMPAR in cognitive diseases has also been recently illustrated by the finding that mutations in GRIA3, which codes for an AMPAR subunit, result in X-linked NSMR (Wu et al., 2007 PNAS 104,18163-18168). Interestingly, mutations in other components of the RAS-ERK pathway can cause syndromes that are characterized by learning disabilities, further highlighting the involvement of this signalling pathway in human cognitive processes (Aoki et al., 2008 Hum Mutat 29,992-1006).

Disruption of SYNGAP1 appears to be associated with a homogeneous clinical phenotype that is characterized by moderate MR with severe language impairment. The absence of specific dysmorphic features and growth abnormalities in these patients is consistent with the fact that SYNGAP1 is specifically expressed in the brain. Interestingly, two of the patients described here were treated for generalized forms of mild epilepsy. Disruption of SYNGAP1 could predispose to seizures by increasing the recruitment of AMPAR at post-synaptic glutamatergic synapses, resulting in increased excitatory synaptic transmission, as has been described in Syngap1 mutant mice (Kim et al., 2005 Neuron 46,745-760; Rumbaugh et al., 2006 PNAS 103,4344-4351). The fact that the epilepsy of both patients was well controlled by topiramate or valproate is consistent with this hypothesis. Indeed, topiramate inhibits AMPAR activity while valproate reduces the level of GluR1 at hippocampal synapses, and, therefore, reduces AMPAR activity (Skradski and White, 2000 Epilepsia 41 Suppl 1,S45-47; Du et al., 2004 J Neurosci 24,6578-6589). The identification of NSMR genes that act along well-characterized synaptic pathways thus offers the possibility of developing reasoned pharmacological treatments that would not only target associated complications, such as epilepsy, but could also aim at improving cognitive processes. In addition, current therapeutic approaches aimed at allowing the complete translation and production of a normal protein in a fraction of mRNAs bearing nonsense mutations would be relevant for at least two of our reported cases, and underscores the value of identification of the precise molecular defects in NSMR (Welch et al., 2007 Nature 447,87-91).

A candidate gene approach that is based on the characterization of de novo copy number changes has recently been shown to be fruitful for the exploration of other neurodevelopmental disorders (Jamain et al., 2003 Nat Genet 34,27-29; Durand et al., 2007 Nat Genet 39,25-27). Copy number changes involving SYNGAP1 in MR, however, have not yet been reported in accessible databases. The candidate synaptic gene approach used herein thus provides a complementary paradigm for the identification of genes involved in NSMR and in other neurodevelopmental disorders. To our knowledge, SYNGAP1 is the first example of an autosomal dominant NSMR gene. The high prevalence of de novo SYNGAP1 mutations in our cohort raises the possibility that disruption of this gene is a common cause of NSMR.

Headings are included herein for reference and to aid in locating certain sections These headings are not intended to limit the scope of the concepts described therein under, and these concepts may have applicability in other sections throughout the entire specification Thus, the present invention is not intended to be limited to the embodiments shown herein but is to be accorded the widest scope consistent with the principles and novel features disclosed herein.

It is understood that the examples and embodiments described herein are for illustrative purposes only and that various modifications or changes in light thereof will be suggested to persons skilled in the art and are to be included within the present invention and scope of the appended claims.

1. A method of diagnosing mental retardation (MR) in a human subject, comprising assaying a biological sample from said human subject for detecting the presence or absence of a pathogenic Syngap1 dysfunction.

2. The method of claim 1, wherein said pathogenic Syngap1 dysfunction comprises a pathogenic mutation in a Syngap1 gene comprising SEQ ID NO:7.

3. The method of claim 1, wherein presence of a pathogenic Syngap1 dysfunction is characterized by a de novo genomic mutation in Syngap1.

4. The method of claim 3, wherein said de novo genomic mutation is a nonsense mutation or a frameshift mutation.

5. The method of claim 3, wherein said de novo genomic mutation is a heterologous mutation.

6. The method of claim 1, wherein said dysfunction is a truncating mutation causing expression of a truncated Syngap1 protein, and wherein said truncated Syngap1 protein comprises an amino acid sequence other than SEQ ID NO:2, SEQ ID NO:4, or SEQ ID NO:6.

7. The method of claim 6, wherein said truncated Syngap1 protein comprises an amino acid sequence selected from the group consisting of SEQ ID NO:8, SEQ ID NO:9 and SEQ ID NO:10.

8. The method of claim 1, wherein assaying said biological sample comprises sequencing nucleic acids obtained from said subject, and wherein said nucleic acids comprise at least a portion of a Syngap1 gene as set forth in SEQ ID NO:7.

9. The method of claim 1, wherein said assaying comprises:
(a) obtaining from said human subject a biological sample comprising genomic DNA;
(b) sequencing said genomic DNA for obtaining a sequence of one or more regions responsible in expression of Syngap1; and
(c) comparing the sequence obtained at (b) with a corresponding control sequence from an unaffected individual;
whereby said comparison allows identification of the presence or absence of a pathogenic Syngap1 genomic mutation.

10. A method for diagnosing non-syndromic mental retardation (NSMR) in a human subject, comprising detecting in a nucleic acid sample obtained from said subject the presence or absence of a de novo pathogenic mutation in a Syngap1 gene comprising SEQ ID NO:7.

11. The method of claim 10, wherein in an unaffected subject, said Syngap1 gene encodes a Syngap1 protein comprising an amino acid sequence according to SEQ ID NO:2, SEQ ID NO:4, or SEQ ID NO:6.

12. The method of claim 10, wherein said detecting comprises sequencing DNA or RNA.

13. The method of claim 10, wherein said de novo pathogenic mutation is a nonsense mutation or a frameshift mutation.

14. The method of claim 10, wherein said de novo pathogenic mutation is a heterologous mutation.

15. An isolated truncated Syngap1 protein comprising an amino acid sequence selected from the group consisting of SEQ ID NO:8, SEQ ID NO:9 and SEQ ID NO:10.

16. A monoclonal or polyclonal antibody, wherein said antibody:
binds with specificity to a truncated Syngap1 protein comprising an amino acid sequence selected from the group consisting of SEQ ID NO:8, SEQ ID NO:9 and SEQ ID NO:10; and
does not bind to a non-truncated Syngap1 protein comprising an amino acid sequence according to SEQ ID NO:2, SEQ ID NO:4 or SEQ ID NO:6.

17. A solid support comprising: (i) a nucleic acid probe specific for identifying a genomic mutation in a Syngap1 gene comprising SEQ ID NO:7; and/or (ii) an monoclonal or polyclonal antibody as defined in claim 16.

18. A nucleic acid probe, wherein said probe hybridizes specifically to a nucleic acid molecule comprising a pathogenic mutation in a Syngap1 gene of SEQ. ID NO:7, or to a complementary strand of said nucleic acid molecule.

19. A kit for detecting the presence or absence of a mutant Syngap1 nucleic acid molecule or protein in a biological sample, the kit comprising a user manual or instructions and at least one of:
(i) a nucleic acid probe hybridizing specifically to a nucleic acid molecule comprising a pathogenic mutation in a Syngap1 gene comprising SEQ ID NO:7;
(ii) a nucleic acid probe hybridizing specifically to a complementary strand of the nucleic acid molecule according to (i);
(iii) a monoclonal or polyclonal antibody as defined in claim 16; and
(iv) a compound for measuring the amount and/or activity of a Syngap1 protein in said biological sample.

20. A screening method for identifying suitable drugs for restoring Syngap1 function, comprising contacting a cell or animal having a pathogenic Syngap1 dysfunction with a compound to be tested; and assessing activity of said compound on Syngap1 activity and/or levels.


SEQUENCE LISTING (56 sequences)

SEQ ID NO: 1 - DNA, 6011 bp, Homo sapiens
  CDS (196)..(4227)
tctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctccatcgaccttcagtccttcatggctcgaggcctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttcttttaa

SEQ ID NO: 2 - protein, 1343 aa, Homo sapiens [sequence omitted]

SEQ ID NO: 3 - DNA, 5962 bp, Homo sapiens
  CDS (196)..(4074)
tctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttcttttaa

SEQ ID NO: 4 - protein, 1292 aa, Homo sapiens
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDREYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQRGSFPPWVQQTRV

SEQ ID NO: 5 - DNA, 6043 bp, Homo sapiens
  CDS (196)..(4053)
tctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcatggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctccatcgaccttcagtccttcatggctcgaggcctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcagcccgtcccttcaggctgatgctggtggaggaggagctgcgccgggaccaccccgccatggctgagccgctgccagaacccaagaagaggctgctcgacgctcagagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttgggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttcttttaagaaaaaaaaaaaaaaaaaaa

SEQ ID NO: 6 - protein, 1285 aa, Homo sapiens
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGSPSLQADAGGGGAAPGPPRHG

SEQ ID NO: 7 - DNA, 33620 bp, Homo sapiens
tctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagaggtacgtggtggggggagggggagggccatatggggggcaacagggggaggggcagggggcgggggagagtcggggccgagggaagggagcggctgaaatggagggagaggggaggaccgggaaggcaggggtgggagcaaccgggaaggagggattggaggccagagggataggggtagataagggatggaggggccaggaaatgggccttgaaagggggctgaagggaaaatgatgaaggggaaagaaagcgccagaaaggggtgggaaggtagaaggattgggttggggaaaaagctgcagaaataatttgggggtgggagctgagtgatgaggaaaacatcaaggaaatagagggacagagtcttgggagaggggctaggaaggagaaatgaaaacataggggtgaaaagagaagccaagaagatgtggggggataatttggtgggggactcagagtatggtaaggggcggaaaaagagaggaggatggagggagagaaggggccagatggagagagagggaggcagtggggggtggggggatggagggtccagatggagggaagagagaagagagagggaacggtgggggaggggagaccagggcagggggagggccaaattatgtgtgcttgggggggtggcagtacaagtaagatgaaatgatgggagtaggggacaggttaggatcctgccccagactctgaccctcttaaaggctcttgaccagtaaaatgtaacctctggggtcttaatgctgtcttcacttcatcacttttacccccttcccccacccctccaatccttatttccattccccctccctcctctgctgtccatcacctatatctttccccccatatggtgtccgttccttatatgagtcccccttctgtccttcccctatatcagtcccctctctggtgttcattgcctctctcttatctcttttctatgtggtcgctcccttatatctctatcacttctttactagtccgtcttgaccccttctcatagcctccgcattaggtctgcctcctttgctctgacacatgtccagtcgagcccttcaggtgtattcccttgcccctgggcatttaccatcatttctgcctccttttctatctactcctattatttgtctcccagaccattccttgtcaccacaagtctgtgcctacccactttcccttaatacctcaactcttattatcatttctcttttcttgtctcctctacttattggggtgttggagatttggggtacaaagacacaggttttgatgattagaacaagggatccccatctcagcccttgagggatttctcatcctagtaaaaatttcccgtttctcagccatgttgctctgcctcttgcatctgcctcccagaatcattcttcaccaagtccagacagtatgtgtgtgtgtgtttctagagttgttcctaagttccctgctgccaggagaaagatgccctctgaagtctaacccgcctcccttggctgcagcctgaaccagccctttccccaaatgcttatgtgtatcaacagatttctcctctgggtataccaagtgtctaagtgcggtgggcatgttgcctgcaatgtctgtagtttcagaggctaggtattggtgatctctgtatctagagaacattagttagtctctgagatgggttgagatagagtttcaaagtatgtggtttctaaatggtggacattctgagggttcatcgctcactgtgatctggaggttgggtaaatttgagagacatatttatagacagaaaagggctgaatttgcaaaactctgctactcagtgcattctgttggaatgaagctcaaagaccagttctcctgcagtatactcatggttatcccataaaggtgacagagtttgatcttttttccatctaaaatctgataccttaagcctaatttctagctctttgctgtcgccaatgtgacatctctcttacttcaatagggcctgtgtggcattggggtttaaaggagaggcagtttgcagggagagcggcatggcaagaacaacagagctggggaaggaggaccctggtctcatctcagccataggcgacacttggtctactgttctctcttctcaaagttttgttcccatctccagctcttgtctttggaggcccctctgtgtcaccaggcctggatattccattctttccccctgacatctttcttcctcaagctcctcactgtgccttgagaaggctcctgacctatggagtggcttccatcctgtccactgagactaggtggggtgaggtggtgggggcagtgttgaatctctgggcactgaatgggagaagagataaatttggtctctacttgtccaaacaagggcatatttcccaccttctgccctggggcctccacagccagatgccgacaaaagccatcttctccttaccaccagaagaaccagaactttccctaaaggatgagcagggttgaactaggggaggagggcacagaggagggaagagttgggaggggaggccctctcatgtctctgactgtgaacaggtaagctccccgacttcttcttttctagatatttagccctcagaatctcatgatcccaatgttcctacacacacaccccatccttctttcccacagccatgtagctttcctcatactcttttatagaggggtggtgccaccttccaagtccttgtcttctgcagctatttggatgccttcctctacccagatatctgacaccaacttctctgtttttctcctgccaattttgcggcacctctaccttattttcctgaagtagatggagagctccttcatctttctctgcccccaaacgtgtaccttctctgtcatataattcccatccctgaacctctgcatttcttcccccaagtctccagagccttctgtgatgtgatcctctcctcttttcccctttgtataaatgcttgtcttgtgttcctttagagttgccaggtgtctcactttcaactgtgatctcctgaaggccggtagggtccccttctcactttgtggactctcttcttgccattttaggcctctgcttccaaatgcatagagcctcccttactgtttctgtgtgtctctgtcctccagatgtacggggaccctctatgcaccgaacccaatacgttcattccccgtatgatcgtcctggttggaaccctcggttctgcatcatctcggggaaccagctgctcatgctggatgaggatgaggtgagtgtggtgagaaggctgggaaaacgcatgtgggagaatggggagagtcctcattagtgaaggggagagagacacaaagaaggcagacaaagaagaaacatttgcaaatggcaggagggtggccattgagacttgggttggagtgtgacaagaggggtagtcattctctcatcttgggcagggagtctttgagggttatgagctgtgttggggaggagatgtggttctgaagatttcgggggtggggaggctcctgttttcacactgtccatattgtaggataggttagaatggggaaggggaactgaagggtttggagaaggggtgaagacatggaaggccctcccaaccaaagcaatcctcaggacccctctctgctttgcccactgagcaagactcccagtcctttctccatcagggggcagagtgcaagaagagaaattgcaggacgcagagaaaaggcaggggatagaggaggttttaggtagctggaggatactgggaaagaagactgaggtcagtggatcttggcatgatggggtgaagactgggagagtgattgagatcaaggtttagagagacactgagagggcttctgcaagggagtggttggacatcaggaaggaatataagggaggggaactgggagtatgtggagttatctaaaggagtacaagtggagtaggggcagggactagccagagtaaaggagatgaaacggacagaggcctcttatgggaggtggtggccaggtatggagatctgagagccagggaagtgtgggttctgatttgggagctgggtcagcaggagtgtccattggaggaaatagaggataaaaactggaatggtgccccacaagtgtgagtcagggaatgggggtgatttgggggacaagctttaaaaaatggtagggtagcctggaaaaccaaacgtggtgagagatggaccctgaaagttattgggaatggtgtgatgaggaagggttagaatttgagtcagggctaactagagtgtcaagggacctatgggggcaggtgagagtgtggagagatggctgggtagacagcttccaggccacagagtaaagtagaggtgttaaggagaggatatggtgactttgaaaggagaggtggcttggggagtctaagttttggacaccattgggagacatttgagtgactgtggtcaaacccctagatcaaggtttggagtctgtaggagaaatgggggaaactggaacccagagtggggactgttatagggatgaagagggaagacaatggattggggtaatgaaggaatgacagagatagggacagactgaagggaccagtggccagcaagttgaggggcaataggaatggccggtctggagcaggaagggcgtgtgtgtgggaggaggagtagatagaattgggaaaatgagcagcctgaacagacgcagggagttggaattggatgggtggtggaactggggctgggggatagtgaggggtctgttcctgtggctctttgtagaggtggaggagactgcaggggtggaggtgtatggaggtagggatggaggtggacagggggagctgggtgtgaagcttagggaaaggtgatggctgaggggaagaagggaactgagagatgctgggtcgggtgatggaaggagatgaaaggtgggggaaggtgaccaggccctctggcgggaggggaggttgggtcccagtctggccgcaagccgggccgtgggagggagacaagctgtggatcctgattataaatgcagcttctgctacccccgtgacaggctcggagggagagaagggggtaagggaggtgggaggttgggaggaaagcagggagaatggaggaactgaaggtcaagggaaaactggattcaggaagtagctaacttctgaggagttgcagaagagctgagtagggtttgggagaattctccggaaggtgtttcaagacaaatggagggagaaaagagggtgggacggcaaagaatgagagagagagactgggggagagaagggagagatccagtcaccaagtgtggaggggccggggcagaaaaagtgggcaggcttagggggaaaaggaggcccgcccttcctgggaggaggcggaggggggaagagggaggggaggggcggcgtgggccagggaggtctgacacacccccacctcccctagatacaccccctactgatccgggaccggaggagcgagtccagtcgcaacaaactgctgagacgcacagtctccgtgccggtggaggggcggccccacggcgagcatggtacggcggccgagcaggctctcatgacccggacaggcgcggggagagggctgaagatggaccgggctggcggggcgggggcgtcggggagcggcgggagctggccgggggcgggagaaggcggagacaaagtcttgcttcccgaggggagatggcgccctcctctggaaggtggagcgggacaggggctgtgggtgagaggcccctgtgggtcctcgctcggcttcgctccctttctcctctccagcagtgaagtttgactattttcctggtgcttgtaaaaatagtctccttagatctttagcttccagtccgtgtgttcctgcccctgcccctcccagcccctggtctgggatcttggaaagaatgacctagttcctgccctccccccagacctcttccccacccctatgctcctgattcttcttccggaccttcagtccttgaggtctccttttattaccccctgttttctcctctcatcagctgcttctattttctgcaattgtatcaccctttctctttcctccctgatttctcttattttctgtacccctcttgattaccccgtccctcccctctgctcccggcgaaagtccatcagactcccatctcatctcctttctgtctgtagagctatctgcgcggtctgtctctccaactctctccctctctccatctgtctcttcccctccctccccctttctgcccccccaaccccctctttagtcatttaaaaaaagatttttgtcttcctgagaagttcgcttaaaaggtttccatgggaacagtgaggggagatgaaggcagagaacagagatctgcagccaaggtgcagagagacgccctcagcccccggaaaccccctcgtcactgcccccttccacttcatttcccttcccggacctagcgatccaggttccccattcctccaacccccaggtgacccctttttcccaccagctccccctcccttcaggaattcaagctaccagtttctctcccttagagagccagtgacctgtccccatcctttggctccttccagccttctgataggtccaggccctactagccttgtgggccggtttggggaccttgggtgggtgggtgggtggtcagggatgtgtgtcattgtcatggaaaccccataccatcggggtgtgtgggtagcacatctctgtgaggcaaagtcaggtggtggtggagtgtatacagagaatgtgtgtgatgtctgtcacatctgtgtgtatatgtatatgtgtgtatgtcatgttcatgtctggggaagatgtgtgtgtttgtgtgcatgtgtgtacacctgcgtttccaggccagaatggtcacatgctcacagagcatgtttatcatgtgtcctgtggttggcagatctgcccacttattccccatctttctccttatcctgagtcctcagggtctaggggtctctggcgggggtctcccgtgtgttttgggtggggacaccgtgtgtgtttggagttgaggtggcatgtggaggttgctgtattggggttcagtaggggctgggaggggttccaggggcatatgtatttggggggagggcttagtgaaagttgatgggggtgaacgtttagggtcttcaggcagacatggaatgtgtaaagggcatggaggatgtgcagtggacataggggtcgttgggaccttggggacttggcaatgccaaggtgtttgctgaggctgtggactctccagcccgggagaggtcgccggacctttgaggggcattggaatcctgggctcctcctctgctgggtggagccgcgaaccctcgttctctccgcggttttgtgtgtgtgggggggtcctgctcaggaggggatggtgggtggcctggtttgttttgggggtccccttgcccccctcccccgtctctctcgcgctgtctccgggcgacagggctcgtgacagaggcggccacggcagcagcggtcgcctagcaacggcggcggggcccgggcaacggcggcagcggcgggagcggcggcggagggagccgttcccgcggccgtcaccgcgcggccgtcccggccgtcccgggccccgccgccgcccccaccgcgccgggcggcgcgagccgggccgtaccgccccctctccctccgcaccccgcccttcccccgcgcaggggcggggcccctcccccaccggggacacccctggcccccccggggggcggtgcgaggtaagggcggggccgggcggactaggagcgcgcgtagaggggggggcgccgcgcgcgtggggcgggggcgcgcgtgtgcgtgggcgcggggaggggggtggggaagccgcggtggcggcggcggcggcggcagctgctccctccgcatgttctcgctgcatcttccgagtggggggagttatggtaaccggagggggacagcggaggacgggagggtcggggaccggcctcgtgcggacagggtcttgagcacgcgggggcggccggtgtgtgtgggatcttggagcccctggtggaaggggcagcccgtgtgtccgcgtgtgtgttcgtgcccatgctcgtgtctcctgtgtgtcggtgccatgtgcacacgggggcccagttcctcgtgcacacatgtgcagccatccttgtgcaagtgtgtcctgtcttatcttgcctctgcctgtttcctgcctagggggctgttgggccagccacgggcttgtggggcagtccgcggccttgctggtccatgtcacctctgaccctgagcatgcatatccctggctggggctcacctcctgtcaggcttctgtgtgtgggaatgtgtggccctatgagcaggattttctgtgtccctggtggcaggtgtgtgtctttgcatgtgtgttccagcactggcctgagtatctgtgtcaatcccagcatgggtgtccagggcgtgtctggatgtgggtggtgtggctggggggagtaagtgtgtgaatgtgttccttggaattctactgtgtatgaaacacctttgggctgggcaggtgtaagccctgcttggtcagtaagtggggacagaaaagcttaaaaagggaagaaatggggtaaagaggaaaagtggggaggggataggagagaaggaatgagagaggcttgaagaagagagcagagagcagcaggccagggagaaagaatagctggagaatgggaatgtgggagaaagccaagccagacttagaagagagaacaggggataagagcttaagatggggaactggatagagatggtgggagaagaaggggtctgggagctggggaggcagtggggaggagagagtcaggccttctggtaaagtgatctctgaaagaagaaagggaagaagatgggagaaggcactggattcttgtaggccttgcaggatcaggcttggttttggatcagggaaggccactcaggcatacgggctggctcacagtgaggcctgtcatagcagggactgtcattagaggttcatatccccagcacagagctctgtctcccatttctctgctctgtgtccaaccctgttcttcgcccttctctggcaacctccctcctctggctccccttgagagctgccagctgggtgtggagaggaagaaaggccaaagggaaggttagggatttggagggcagggggagattctgtgggtgatggcaagattagagatgttggaggggcaggagtccaagcccttgctgtgtgtgtgtgagtgccattgtgtgtgtacacagggtgtgtacatgtgtgggtgtgtgcacaatgcctgtgttacgggtttgtgtaggtggctgttatgtgtgtgcatgcgtgtatacatcgtgcaggcatgggacatggagacccaggatcacagtttgtgcttttcaggtttgctgtgtgtgtgtgtacacgggtgtgtgttgttttctctgtacatgtcactgcatgcagcattaataacaggatgttctgtgtttctgtcactctgccccccctttcctctgtgtttctgctattcttttcctgcggcaacaaaagtgtttaataagcatatgtgtatgtggcctctgtgtacctgtgacttatgagtgagttcatggatcttagttcttggtaatatgtgtaaggttatattacttccaagaaccaagcaagccaattagcaaatttgttcttctgttaatgctttagttccaaagcggaattttaatgcatttaaagttagactttatgcctttattagatgagcttgcacatgaaaataatggctgagctcggttccctgaaatgagagtgaggcagccatacctgagaaatgcagaaaaaatgttactagggaagagatatcctgaaccattggatgcattgcttagtgtgtaccagaaggataattaacacatttgctcctggtttttctttactctggtggcaatctcggatgcctgtgttagagtgagagaagggctgggggaggaaagggggttgtcctgagacagagtggatgtggttgtacttttcacgtgaatgaaaggatgtttgtgcttctgagatatggggatacctttctgtggtcaggatcatggtgtactcaagcatgtatttcaggggttaaactggaattcccctgcatggggatatgtgtgtcttagggttataggtccatttctgtagtgtgctgtccacatgattgcccaagagacttgggccacaaagtccacaccagcagctctcatgtgggtcactgggttcaagtatgtactcccttgttgcagagctcacatatgtcaaagcatgtatcctgtggggtgtgtgggcctcagggtctcagaatgtggatccgtgctatgcccgtgctcacagttgtctgtatgtctcaaaagcacatagatccccagttatttttctgtattgtggttctcatatacacgtacctccatagctcagtatatgtttctgtactatacacctgttcctgagggagtgatagggttctcgtgtcatggggtccacatttttgtatgcaaacctcctaacacctgggttttacaggtaaagggaagctgaggactgattctagggcagtttgcaggcaatttgcaattctggaagcagacagtgaaaatataattgtggtcctcccttgttctgttctggaacctagagggttaacaggcagctcttccggtccacccccctccttccagcctgtagcttcttgttgccgtggagatggtggccccgccccgatgcagcacctgcccccccattggctgcagtggtgactgtggggctgaggggggagcccaggcctgggcagccattctggagctggagccggagcttggcatccccccacttacattctcttccagcccctcggcccctctaaattcccttgcagccgagcctcagcttcctttttcagcgcttgccatccctctaggctcttggcagactgagctgcatccgcattcacctcccctcctcctcctttccctatccttacttgggagtcccgagaacccccactgtccatccttcctgctccctgcccattcccctgcattctttgcttttcccctccccccaccaaccttctgagcccctgccactgcagtgcacgtggaaccggctccttctcccttccccccacctgccccttccttggattttctgtccccagcttctccccctgctactcttcacaggtctcttccaagacctccccctccagtccagggagggggtcatgggaggcagccctggccctccagacagacaggggttccaggaggtggggggcaggtggaagatgtgcccaccagggggaagaggaaagcctgcctatgggcaatgacctttaacccatctttccccccagctagccccttctggggtgggtgggcaccagggacctagcctccctggatctttggtgtccttcattactggggttttactgaccctctcagccatgctcccctgctgactgccagccccagtcatgggcctaaggcctcccaccccatccccctcagggggctcctgctcaggttccttgccccctccttcccgctgccagcctctccgccgtcgctgctcttcctgctgctttccggggggtaggtgaggccggagctgaggggcagagggaggtgggggcatgcactggggtcaggggtgggaacctgggttaacagcttcccttctggccccctccccaacttcccttctggccatttcctcccctccagaataccacttgggtcgctcgaggaggaagagtgtcccaggggggaagcagtacagcatggagggtgcccctgctgcgcccttccggccctcggtgagtggtgcctaccagatgtggctcagttgggccccctcccctccagccccaactggggccctaggagtctgagaaagagggcaggagggggagagagggagtgagagtgagaaaaagagtgtgtgtctgtgtgtgtctccccacctctttggtttcccccttcttggctctgcccccctgcttctgagaccagccctcccaccttctccaagctgtgtgtgtgtgtatatgtgtgtgtgtgtgtgcttgcactgtacccgtgggagcaaggaagacaaactggtattagggttcctatccccctttcctcgcctctggagacttcccttttccccatcccactttcatccaggggctctctaccagctgagggatgagtaggtagaactgaccctgccccaacccaccccatccccatttcccccccagcaaggcttcctgagccgacggctaaaaagctccatcaaacgaacgaagtcacaacccaaacttgaccggaccagcagctttcgccagatcctgcctcgcttccgaagtgctgaccatgaccggtacaggggctggagcatgtgggatgagattgatgtaatgtagggtctcctgtgtgagatgcagagggagggggttatctgtgtgcaaaggttgaaggattcaactcaagttggttgggggatgtcatggcacaggggacagaacagaaaagaactagaatagggatctgtgagcagcaggagaggggtagggtggcagagagaagacagacagacaggctggaaagggaatgaaggtgaagccaaggagggactcctcagggactcctcaggccaagaaggatgggctctagcccaggatcaaaggagctgtacaggaggagagtgaccctggaggaatgtttaaggaatgcagggaaggggttggtaggtgagtgagcaataggctgtaggtggaagggtgtcagggaaggtcaggaaatacaggggcagcaggttggagtggggctgggggtggctgaatgaatggatgatggctagggctcaaggacctcatcagtgagggaagagacagtatagagcatggcagagaaggggaggctgggacaggtgtgcagggtgacagaatgggaagcaacccatggactgaggcatgaagaagcagccagcggagaagtccagaaggcactgtccctgagaccaggctgaaggagacctccactgtttgcctttgttgcctgccatttggggttcctctctgggtttccccctcacccagtcactccccagggagaaccatgccctccctttcccccatgtctggccacccccaggattgggcaggtagggaggttgggataaagtgagtcacacctttccctgcccccctcccatgttgccagagctggatttggggccggcagggggtgagggcatggtattcctggccgcgggggcgggggggggggtccgggggccgggggagcgtcgcgctgacggcagccagagcctgcgatgacggggctgctataaataacttcttggaggctcccacacccaagctcccctcccgctttcccactgctctctactcttcatcccctgcccatctccataccgcttttgtattgctatcctacccctcattattccatgcccctagccccctttatcttctgccctcctgcagtgatttttttgcattccatcccctcttagccctcacctcggttctcccggccatctctccagttggccttcctcctcttctcctgtcctctgtcttgctgcacatacctttgtctccccctttcttcttcttgccctacctcctcttcttccctagtccgtgtattctgtcttttatcctctttgagctcttttctgcccacagctttctcctatttcttatgcttttccctcactctttcccctgcttctgctaaaacttgtcctcttatgctgtgttcattcattctttgaatcattaaatgtttatcaggcactagccgtgtgccaggcccaggctagacatatctcttctctgtgccttcacttctttacttccactttttcctttatactgaggctctggtttctggggttacctggaggtactacctagaagtgccccaggcccactttgttctctccttttttttttttcttttctgccatggtccatttctgggttgagatatttctagatgtccccagtcctcgcaatcccttaggtgtgagatggtgggagtttcttttttttcctttttttttttttaaatagaaatagggtctcactgtgttgcccagactggtcttgaactcctgggctcaagtgaccctcccacctcggcctttgaaatgttgggattacaggtgtgagccaccaggcccaggtggagcaggggagttccttaaaggattctgatttttctcacatccctcacgtccttcctgataggcagggtttctttctgtgtctgtttgggaagggtgttcagggggccttctctccaagtctccatcctggaacagactgatgatgcagggtacctatgtgtctaagaagagtaggggggccgggcgcggtggctcatgcctgtaatcccagcactttgggaggctgaatcacttgaggtcaggagtttgagaccagcctgaccaacagggtgaaaccccgtctcagctaaaaatacaaaaaaaaaaaagaaaaaaaaattagctgggtgtgctgaggcaggagagacgcttgagcccaggaggcagaagttgcagcaagccgagatcacaccactgtactccagcctgggcgacagagcaagactgtctcaaaaaaaaaaaaaaaaaaaaaaaaggaagagtgggaagccctgatcccttcctctcctgaacctcctgcctgccagggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtactgggtctggtgggctggggagggccaaaggacaggggtgatggaaggtggggggcagagaggtctagagaaagtggcacaggtggggacatcaaggaaacagaaacttctgggactggaggaaagggtaggccagggaggaagagaaggtagcagagtctcctcccctctgtagccctttccctcaactccacactcctttctaggtaacaacttcatcaggaacaaaatgctttgcctgtcggtctgcggccgaaagagacaaatggattgagaatctgcagcgggcagtaaagcccaacaaggtattggggaataaaggggacacaacctgtgcagggcaaaggttgcaccaacacgggaagctgggggcctagggaggaaagtgagttaaaggaggagaggcttggggaaggagaggattgaggtacagtgtatctggacaagcagggggagacccccattattctgagtcccccatttcttttcgctttctgtactgctaccctgccttacgatctctttccctgccatagaagtcatagacttacagagttgatggggctctggaaattctttagtctagcttccctgcaggcaggaatgcttcactaataccccgcaggacacatcaaatacacttagccaagtttctgtaccttggtttcctctctaagacagagggaactggtggcaatgggttggattaaatgatctctaaggtccctttggcacataaattctatgagtctgttcttcccaaaacttcatgcttccagttctgtcagctattctctgtatcccagtttctagcaagcttacagtcctagtcacactcctctggggggaactcttgtgtcttgatgtccttaaagaactcaacccaagatgctgacatgatctgacctatgcagagtacaaacaccatgttccctttcaacacactgcaatatcacctgagccttaccattagcttgtgctcaattgtatccccccaggcccttactgattcttttttttttttttttgagacagagtctcactctgtcacccaggctggagtgcagtggcgtgatcttggctcactgcaacctccgcctcctgggtttaagcaattcctgtgcctcagcctccccagtagctgggattacaggcatgcaccaccacacccgactaatttttgtatttttagtagagacgaggtttcaccatgttggccaggctagtctcaaactcctgacctcaggttgtctgcctgcctcagcctcccaaagtgctaggattacaggcatgagccaccacatctggccaagctttacccattctatatgcaattcttttttcacaatttctgatagtctctgcaggactttccagttcccttcaaattctacattaatatttttggcttgttattccagcttttgaaatctttccaatactgtttgtgttgtctagtgtatgtctacgcttctacagtagcttcctggggctgctataacaaactgctacaaactgggtcacgtaaaacaacagaaatttattctctcacagttcaggaggctggaagtccaaaagcaaggtatcagcagggccacgctctctttgatgctgtagcagggaatccttccttgcctcttcctagcttccggaggttgccagcagtccttggcattcctgggcttataactgcatccgtctaatttctgcctccatcttcatgtagctggcttccttctgtgtgtctctgtatcctgtatctctgtgtctccaaatctccctctccatataaagacaccagtttttataaggtgggttaagggtccactctaattcagtatggcctcttttttttgagatggaatatcgctcttgtttcccaggcttggagtgcaatggcatgagctcagctcactgcaacctccgccttccaggttcaagtgattctcctgcctcagcctcctgagtagctgggattacaggcacgtgccactatgcccagctaatttgtttttgtatttttattagagacggggtttcactatgttggccaggctggctcgaactcctgacctcaggcaatccacccacctcggcctcccaaagtgctgggattacaggaatgagataccatggctggcctcttcttagtttgattacatttgcagggatcctgtttccaaataagatcacattcacaggttcagggtagacatgagttttggtgggatactattcaacccagtacaccgtctcaccttatgattgctgtctgtatcccactttcttactgtctctcctcttttggtgtctctctgtcccttccccctttaacatcatgcccaccccaccatgccaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggtgagtgttgtgccctcagggaaaggtgacttgggaatgggcacttgcttgggggttagtgaggacagggcaaattcacgagattgggttgtgcagaggctgacacttggattttcctgggcctcaggacttcctttcagacatggccatgtctgaggtagaccggttcatggaacgggagcacctcatattccgcgagaacacgcttgccactaaagccatagaagagtatatgagactgattggtcagaaatacctcaaggatgccattggtatggcccacactcaggccctcttcttcccaaacctgccagatgtccaccccagaccccaagtccacccttccacagcttgatacttcctaacccagagtcctaggactccagcctccaacacctgattctgaaatttccccaaccctggccacccccttccctgcccttggaaagtgtgaccacaccctcttgtgcccccaccccccaggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactggtgagactgggaacgctgggctggggggccagggtcgggggaattatgtgttcatctgttcatctatctgtccatcctcaaagaggactgagcaccatttatgggcaaagcattgttctaggcgctatagagcaaacaggtgaaagaggcctggtccctgccctcagagggcctccaccagaatggggacaaattagaagaaaaaaaaaaaaagccacagagccataatggtgtgtaagtgctgagtaagggtccccccaacctctgtgtgacataaggtcagagagaaggcagagctttgagataagtggggaagaggtgcccccttgggtaggctttgaagactggtttaggttctgatatatggacatagttggcaagaaagacatttcagaagaaggctgtgagaaaggcacatgtgtgatggtgaaaaggcccaggagttttcaggggacattaaagtaggttagtagcaattacatcaggtttagtggagcatgtgcctcataatggggagtggcgggagagatgtctgggcaggaagattagctttagaaactggaaggcctcaaggagtctgaggtcattggtaggccttgggatgccattaaaggtgtcagaaatattgtgatttagaagattaatccataggctgggcacggtggctcacacctgtaataccagcactttgggaggctgaggcgggcagaccacctgaggtcaggagtttgagaccaggctgaccaacatggagaaaccctgtctctactaaaaatacaaaattagccagatgtggtggcacatgcctgtaatcccagctattcgggaggctaaggcaggagaatcacttgaatctgggaggtggaggttgtagtgagccgagatcacacgattgcactctagcctgggcaacaagagtgaaactccatctcaaaaaaaaaaaattaatccataagtagagtgtatgtagtagagtagagggatgtatgttgggggatgactggaatagagccagctgggggctcggaggcatgaaagtcagatcctgaatcagaacagtgacagaagttaggaaggagctactggaaggaccctctcgggaaaggatcagtaggaattgtcagcttattggaaaggagggagcatgtctgtggggagtcacggatgactcaagaggccatgaggctggtggttgggagaccggtggcctcattgacaaccaggaaagtcaggaggaggagccagttggaggtgtgggggcgatggtgagctctgctttacaccagccgagtttaaggtgtcagtgggacattgaagtggaactgggaggtgcggggagtgagctctgagccattccagggactggggatcatgcctggggcacctccatccccatttccctggaatccagaagagttggggggtccgagctccctgtacctcaagtgaccctccatctctctcccatctctgtctctccctggtgtctgtttttcttctcctcctctccttgtctctctcccacacccctccatctctctcccacgtgtctctcccctcaccttctctccccctccatttctctctccctaatctgtctgttccctctgccatggcccccttcttcaagcagcctcccatcttgctcctgcggtccctccttccctgtctctctcacccctgtttccacaccctcacctcctaccacccccctcagcatgttccctggaagctgagggtctctggggctcagtcccggtctctctctttctctctctctctctctgtctccccgacccttccccccagcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtgagggaagcttcaggagtgggcagggcagggagtggcagggcagggagtggcagggctgggggtcggcaagaagggtctcctgagtccccagagatcctgagatggggaggctatgataccttgtgtgtgtgtgtatgtgtgtgtatgtgtgtgtgtgtgtgtgtgtgtgtgtatgtgacctttatcttctgcattcttggctaggtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggtcagcagatcccctctttgccctatccccagatggctccagaggttcctggagcctgagaaactaccctttgaagattttttttctccccttgtttctcgaggtgtcaccactactatcccaactcaggccccctccacctgcaccctcagaggccctcttagagctgggcactgagcccccaggtaacagcctcacccttccaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacaggtgagcaccctgggacagccaggcctgtgccctaggagcccttctcctattctagatactcctcactgggccccacatgcatctctctagggcttgaaagaagggaggaaaaagcaccaagttctcaggggagacgataaggagacaggtacagtcagtggtaggctgagagccctttacagcctgagggagtgagagatttggagctctaggaatagggctgaggctccaccaactcacggcttagttgtaagcctagagcatccctgctgcaagctctgatttgctgtccctctgcctgcccatgctagtccccaggctgaggttcagccagcatgtcatgtcagccatgtgtcaaaatgttcaaacatctcagtaatagctagtgaataagcacttcccccagcccccgaccacaaccccacagacctccccatgatccagcacttagagcagtgacagcagaagcctagcagggcctgcagcctgctccagagtcccagcctccattctgataggtggtgcccgtgcttcatttgctgctcattattttgatgatggccctgctttttcctctgcccgtgtcttcctccatgacaccatacccatcccaccattcccgcctctcctttcatttgtccacatctctctccttctctgtctgtgctcgcccctctttccatctctctccagctccatcgaccttcagtccttcatggctcgaggcctcaacaggtgaggggctctcccctcccccgccctcctctcctctcctgtctgttccctctcccactccactggccttcgccctactcctctcctctcctcctccatggacctcatctcctccatatgtgcccagccctgcccccatcccttctcttgctgcccccatctcccctcctctaggcctcacccccttcccggaggggccctgtcctttccctttactcacctgtcccctcccatcctccctgcctgccctcttcagggctgccaccgctagctctcagcccttccctctgggtcccacttttcaccccaaggcctgtgccagaccacagcaaggttcaattgctaggagccctgaccttaccttctgcttgtgtgcccccttcccttctgacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcaggtaggtgaaggcaggaggaaggcgggctgggtcacaacagggagggaagaaggagatggggggtggggttgaaacagagtctgtggcctgaagttacaatcttcttgcctccttttggccattaaacaaatgtgtatagagggcctgctatgtgccatgtgctatgccaggcactaaggatacggcactgaaccctcttagcactctcattccagaggggattaatccataagtagaatgggggtgactggaatagggccagctgggggcttggaggcatgaagatcagatcctgaatcagaacaacaggagaaattctctggcattcaactcacatctctggcattcagaggtgataaaggctacagcagggcaataggactgggactgtcgtctcctttggctgtgctgttgccctctaaatgtaccctgctttttcccaccttttctttctctgttcgccctcactgtgccttgtcccagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtacgatgggctctaccagctccaagccccagtgtttccttttacccacaggggagatctctagtcacttccaagggaaacctccagggtcagttatggtggtaagaaaaggcaaagacctgatcacctcttgagaagccttccgtccatctggggagaccagatacacacagttgttaaaagtcactttcaactctattttgtaggttatatatatcatagactatctggggatcatctattttaggcttacctccatgttcttccccaggaggaacataagccctggctcctgtgactgcttaggaaaggaaatgttactttactggcagtaggagctaataaattggatggggatggagggggttgcagctatattacaagtgatgtctggcctacccagtcactgtcccatgggcataagtgacagtcagtgggcagggaatgcatgtggtaaagtagtatgtatcagtcatgtgtgaaaagaccatccagaccctcacttggtggaggctgagggcaaagggcaccaagagctctggggctaacacagataacttagcagaaagttacttgaggaagctgttgttcctagggcctgaggaagagggcggctgcaggattaggtcatatataaagaacatatgatcctaacacggggaagtggtctttgagcataggaaatgttgggactgactcagatgggcttgaccagcaaatttaatgagaattaaaccctaatattagccccaaggcagccaccgtaggaagttaattcacatctctcctctgcatgtagaagggttggtgatatatgtatgtatcctcttcagagaatgaggaaatagtcttttgaatcatttttttttctgtgcttcagatgaggaaaccttgttgagaaaggctgtagactagcactatccaatataactttctgcagtgatgaaatgttctgtaatctgtagtgttcacatgtgtctagttgtgattcaggaactgaatttttagatctatttaattttaattttgtaatttttttgagacagggtcttgctctgtcacccaggctggagtgcagtggtgcaaacatggctcgaggcaacctccgcctcctgggctcaagacatcctcccacctcagccccctgagtagctaggactacagggtcgtgctaccatgcctggctagttttttttcttttcttttttttttttttttttttttgtagagacggggttttgccatgttgcccaggctggtctagaacttgtgagctcaagtgatctacctgcctcagccttccaaagtgctgggattacaggtgttgagccactgtgcctggcctaaattttaaatagctacatgtgggctggtggctaccatattagacagcacagctgtagactctagagccaatagggcagataaaatgactcttctgagctcttccagctctggaattcctagattctcagcctagaattcacttgtagacctcctatccttggcaccttgacagagccaaggagagggtcacatggaggacagcacctgcctttcccccgtcaattgccttttcttcctatgtctccagcatgtgttctggggcctggcttagggctgaagagtcaccctatttttaatttctgagcacagttactcaaggtgtgtttgtgagtgtaggtttctgtgtgggtcttcatgaggggccatacatagacctcagggtgtgggagtctgtgaccttgctccattttgagggaacctcagcaccatggcagggtcttctcaaaagcaggtgtgtggatatgatccacaggcagggaaggggttggggaaggcagctggatgtccctcaacatgcagtggctgtgcttggcagacagggatggaggctgggtggtgggcttggggtggggcgcccctcatagtgcggggtcgtgtgcccggcgggcaggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggtgaggggcggcctggggagggggttgtgagggagagcctgaggctggagagagcaagtgggtgagctactccgctgactcccatccccaaactcaggagcccaccaggagagcccaccactctcctccccaggaagccacccactcactcatcaccagatggagagaaaccccaacctgcttagtgcattaaatatctctttaccaaaccctgacctctcttctgatagagtagcttcggaagcccttggaaaatgtacctgttcctgtccaaccatcactgcatttgcatttaccctaggccagagctccccactagttattctcaacttaacctgtgatgttcactccaaacctaagcagggctccctagccagagtaggccctgcccttcctgggtggaccctccctctctagccttggaaaggtgttctgttagaaagggtcttttagcctgtgtatgttttcagctgctccagcaagtcctgggctccaaagagggtatcctcagcaaagaggtcaattatcttcagagtggtggggtcggggtgggggggaccctgggcgcactccaaccagagccacctccattttgatccattctaaatgtattttatgtaagatttaattagaagaaaagggcttcttgaaatattttttgaaaaccactgctctaattgatatcctttatgataaatcacctcgaggatcttcacagtgaggtgacatgggggatgcagaagccaggtcctcagccatggaaggtctggggaaggggcactgctgtcctgattgggacgatggaggcctggaggtgtctggatggtagaagtctttgaggcacagaaagctgccttagcagggaggtgtaagggttcctgggaggaaggtggagagcatgatcctgaggaacagggagtcttgcatcacggcaatggagggactctgattctaagggataaggatgcctgaggtttttccagagagctatggggttccatgggcaggctctgagcctgtgcccgccactaaccccactgaagcccgtcccttcaggctgatgctggtggaggaggagctgcgccgggaccaccccgccatggctgagccgctgccagaacccaagaagaggctgctcgacgctcaggtggaaattacaatgtcatttatcttctccgtgtcccatccccatccatcccactgtctttcgtgcactcactacaccagccacctagccccatcaccatctgtctctcatagtctgctgtttgtccactggctgctcctggcagccccctagtgaccccatcttcatcccatcgtctgtgcctttgtcactcctggcagcgtcagcccaactcctgtgccttcccatccagtcttcccactcctctctgcatctcaggaccttctctaccagaaccttggtctttctgcccctagaccccacctagttccaagaacccctgccccttctttgctcactcctattcaagccacgttgttcagcttcctctgcgctcttgggccagagggctagaagctgccgttttctggaatagagcacagggcagtatgatctgtagtttctccaggccctggccggtaccctgaaaacttggggacccatcacctctgttctcttggctccctaattttcctgtctccttggcagctcctgcatagcttcctcttcctgactcttcagatcttgaaggccttccatcctgtaacctccctttgccctcagtatttaagtccagcctccctctggcctccctcccactctggccctcagaccttcccagctgcctgctgcccagcctctcttctcacaagccagcttctaggacctcccttctgcacccttaccccttgctttcccaaaattctgctcattttcctacccatactcctctttgctctgactgctaggctccccccgcctgccatccccccaccaaggctcctgaccccatgaccccactctctcccactgcagctcctcatcaggtaattctcctggttccgctttggccacgggcggaggacacagggggaggtgactccggaccactgcaggttggtcgtgaagcccactccctccaacacctccggagcctctcccctctcactgctgccctccacacccagagaacctccacagactccagccctccgacacctgcacagatccatctcccaagacaccacccaaagagagcatttgctgctgcttcccagaactgtccaacaataccttagcaacaccaagagttgggccctagatgggcccagcacattcacaggtcacacccacttccctgcaaaacccaccccctcccagcctcctcctgactctaagccctcctcttcctctacctctccagtgtatgtctgtcaccccccatttcaccagagcgtccttaggggctgggggtgggtttgttaatggggtggaggcaatgatgggttggaggatcttggctataggggctgtgctgactgcagcaggtaggttgggtttccctcttccttccctaatcttggttctctaccctcctttccactcctcacctgattctctctcttcctcctccttatatctgtgaggcagaaggcatctgaagctcatattagcccccattgggtgggaattaggagtgggtagttaactcagggagacttgagataccctggaaaaaatgctattgagatgtcctgacattaggcagggtggatggaacaagaaggagcaagaaaggaacctcaggcagatgttaggacatggacttgatcatgtggcctgggagtttagaaatggggagagacatcctcctagatcagatcgtgggctcagtaggcatgttgattcccagggagaggtgccaggaacagcatggtaaagaatgtactcttcacagctcacatccccaggttgctgatgccactcactccccctctcctgccatcgagtggccttgccggacacatcaccctacctaaaaagccagtaaatgagaacctgtcagctatagccatcatttctgagatgcgattttctttgggattgagctgcagtgggcagtggctccttacactgtaattttaattctctgcctgcccagcctctctgtcaaagtagctggtgatctataaagatgctaaaaggcaccaggggactttgccatttaaaggactcctgcagtgaattcttttgtaaaatgaataatggcaccctaatttatccactttctaaatttgggtccatgggggtgtccagggcatgcttatgtgctgtcaccagcagacaaacagagggaatggaatctgggggttccttccctgctctcccgccatactcaggataccctaccataagtgatttcctctcactgacttgcagaaaatgtgtgagatacccagcaagctaagaaggcagttttgctgggtatctcatacccaaggctggggtttgggtgatctgagaggttagctccttgatcctaggatggaagggagagcttatatagaagcttttacttggaaggttttgtatcctaaggtcagacatagctatattaccaagcctaaatgccatgtggcccaggaaataatttggacatttgttctaaaccacttgtggtaggtattggtctctctgcaactcagccattaattagaaattagttttgagcctgaattttaaaaagccaagtgttgcccccagcccacacacacacacacggacatgtacagtacaaaccccagataattacaacagccaaagagagaaggaagtgaatttcccaaccagaagcgtagggaaattcagatggctttcttttctcccagcagaggaacagaaggcggagctaagggcaggaaccaggagttggtcaaggagctataggaggtgatgagagtagaaccaggggtaggagctggtctggtacccctcaccctctaattgggagcccagggagaaggactgaaaagaagatgggagtggaaaagaataaagccagtttctgcttcccagggatgcagagattggggcatgctgtgtctgcagaagctcctagtcatttccgccataattgtgagagagaggggcagccctcccacaagatttttcccttcccatatcacttccctgaatccccttcctttccccccagtacagttaaacctctctctaatttggaatgttatatttgagaagatggccactgtgaataagtgacaaacggaatgacagtgtctatttaatgaaatgcatgtcttcaaaatatatacagaactcgatgaacaaggctttttccactcctcagggagcatgcattaatgaatagataggattcaaaagtctgttttctggtatgggttaaatatcccctcctacagacatatttccaccactaatttgcttagtacaccttttcttcacagataaaggaaaatgcaagctcagtttttcttcagattatgaagaaattccaaatccacaggggtttggattaatgaggttttgctgtactgcctccccttattcctcaacatgaagttcccacctcggattggggatgggtgggagggggtttcaagaggaggagggtgggatgggcaaggaatatacacaggtgaagccagagaagggttaggttgggggtgcggtgggaacttgctgttttgatctggtttcctggtgtgacactctgggttaaaggcttgaaggcccctgttaggagtctaggggtgagattctcttctctctgatcccagaggacgttaacttctactgcaggtgagaaacaaaataggaggatggtggggactgtcctgggaggagggggtggtccatggcttgtggtgtgggctggctataggggaggccactgctaggggactggcatccaggcccccttgaagcgtctcaataagtccgcgctctcctttttggtgtcttgcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 8 - protein, 137 aa, Homo sapiens
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRL

SEQ ID NO: 9 - protein, 578 aa, Homo sapiens
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAE

SEQ ID NO: 10 - protein, 834 aa, Homo sapiens
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPRPVPHQHTARAARTSQSQSRRC

SEQ ID NO: 11 - DNA, 21 bp, Artificial
  Sequence is a chemically synthesized primer
ggtctcgagcctccatccatc

SEQ ID NO: 12 - DNA, 24 bp, Artificial
  Sequence is a chemically synthesized primer
ttttccccaacccaatccttctac

SEQ ID NO: 13 - DNA, 20 bp, Artificial
  Sequence is a chemically synthesized primer
cttgccattttaggcctctg

SEQ ID NO: 14 - DNA, 19 bp, Artificial
  Sequence is a chemically synthesized primer
agtctcaatggccaccctc

SEQ ID NO: 15 - DNA, 17 bp, Artificial
  Sequence is a chemically synthesized primer
cttcctgggaggaggcg

SEQ ID NO: 16 - DNA, 17 bp, Artificial
  Sequence is a chemically synthesized primer
cagcccggtccatcttc

SEQ ID NO: 17 - DNA, 19 bp, Artificial
  Sequence is a chemically synthesized primer
gggaacctgggttaacagc

SEQ ID NO: 18 - DNA, 21 bp, Artificial
  Sequence is a chemically synthesized primer
tctttctcagactcctagggc

SEQ ID NO: 19 - DNA, 20 bp, Artificial
  Sequence is a chemically synthesized primer
atccaggggctctctaccag

SEQ ID NO: 20 - DNA, 18 bp, Artificial
  Sequence is a chemically synthesized primer
cccctccctctgcatctc

SEQ ID NO: 21 - DNA, 18 bp, Artificial
  Sequence is a chemically synthesized primer
aagttgcagcaagccgag

SEQ ID NO: 22 - DNA, 20 bp, Artificial
  Sequence is a chemically synthesized primer
cctaccctttcctccagtcc

SEQ ID NO: 23 - DNA, 21 bp, Artificial
  Sequence is a chemically synthesized primer
gggaggaagagaaggtagcag

SEQ ID NO: 24 - DNA, 19 bp, Artificial
  Sequence is a chemically synthesized primer
actttcctccctaggcccc

SEQ ID NO: 25 - DNA, 19 bp, Artificial
  Sequence is a chemically synthesized primer
ttgcagggatcctgtttcc

SEQ ID NO: 26 - DNA, 17 bp, Artificial
  Sequence is a chemically synthesized primer
tgctcgccccagaagac

SEQ ID NO: 27 - DNA, 19 bp, Artificial
  Sequence is a chemically synthesized primer
tactgtgagctctgcctgg

SEQ ID NO: 28 - DNA, 17 bp, Artificial
  Sequence is a chemically synthesized primer
tgctctgtgaagtggcg

SEQ ID NO: 29 - DNA, 20 bp, Artificial
  Sequence is a chemically synthesized primer
gaaggacaaggcaggctatg

SEQ ID NO: 30 - DNA, 19 bp, artificial
  Sequence is a chemically synthesized primer
gccctgtcctcactaaccc

SEQ ID NO: 31 - DNA, 20 bp, Artificial
  Sequence is a chemically synthesized primer
agtgaggacagggcaaattc

SEQ ID NO: 32 - DNA, 19 bp, Artificial
  Sequence is a chemically synthesized primer
aagctgtggaagggtggac

SEQ ID NO: 33 - DNA, 19 bp, Artificial
  Sequence is a chemically synthesized primer
cagatgtccaccccagacc

SEQ ID NO: 34 - DNA, 20 bp, Artificial
  Sequence is a chemically synthesized primer
aatttgtccccattctggtg

SEQ ID NO: 35 - DNA, 20 bp, Artificial
  Sequence is a chemically synthesized primer
ctggaagctgagggtctctg

SEQ ID NO: 36 - DNA, 18 bp, Artificial
  Sequence is a chemically synthesized primer
agacccttcttgccgacc

SEQ ID NO: 37 - DNA, 21 bp, Artificial
  Sequence is a chemically synthesized primer
gggaggctatgataccttgtg

SEQ ID NO: 38 - DNA, 20 bp, Artificial
  Sequence is a chemically synthesized primer
agggtagtttctcaggctcc

SEQ ID NO: 39 - DNA, 19 bp, Artificial
  Sequence is a chemically synthesized primer
ctatcccaactcaggcccc

SEQ ID NO: 40 - DNA, 19 bp, Artificial
  Sequence is a chemically synthesized primer
gggcccagtgaggagtatc

SEQ ID NO: 41 - DNA, 19 bp, Artificial
  Sequence is a chemically synthesized primer
ccgcctctcctttcatttg

SEQ ID NO: 42 - DNA, 19 bp, Artificial
  Sequence is a chemically synthesized primer
agaggagtagggcgaaggc

SEQ ID NO: 43 - DNA, 19 bp, Artificial
  Sequence is a chemically synthesized primer
ccagaccacagcaaggttc

SEQ ID NO: 44 - DNA, 20 bp, Artificial
  Sequence is a chemically synthesized primer
tctgtggtgacacccatctg

SEQ ID NO: 45 - DNA, 17 bp, Artificial
  Sequence is a chemically synthesized primer
cgctgacagcagccttg

SEQ ID NO: 46 - DNA, 18 bp, Artificial
  Sequence is a chemically synthesized primer
agcatgtgctgcaggttg

SEQ ID NO: 47 - DNA, 24 bp, Artificial
  Sequence is a chemically synthesized primer
ccccctgctgcctccatccttcat

SEQ ID NO: 48 - DNA, 23 bp, Artificial
  Sequence is a chemically synthesized primer
aagcccccagctggccctattcc

SEQ ID NO: 49 - DNA, 19 bp, Artificial
  Sequence is a chemically synthesized primer
gtctcctttggctgtgctg

SEQ ID NO: 50 - DNA, 22 bp, Artificial
  Sequence is a chemically synthesized primer
ggaagtgactagagatctcccc

SEQ ID NO: 51 - DNA, 17 bp, Artificial
  Sequence is a chemically synthesized primer
acagggatggaggctgg

SEQ ID NO: 52 - DNA, 18 bp, Artificial
  Sequence is a chemically synthesized primer
tttggggatgggagtcag

SEQ ID NO: 53 - DNA, 20 bp, Artificial
  Sequence is a chemically synthesized primer
tccagagagctatggggttc

SEQ ID NO: 54 - DNA, 20 bp, Artificial
  Sequence is a chemically synthesized primer
gctaggtggctggtgtagtg

SEQ ID NO: 55 - DNA, 19 bp, Artificial
  Sequence is a chemically synthesized primer
ctataggggaggccactgc

SEQ ID NO: 56 - DNA, 20 bp, Artificial
  Sequence is a chemically synthesized primer
atgtccaatcctggtggttg